(19) United States
(12) Patent Application Publication
(10) Pub. No.: US 2019/0336610 A1
(43) Pub. Date: Nov. 7, 2019

(54) GLUCOSE-SENSITIVE PEPTIDE HORMONES

(71) Applicant: GUBRA APS, Hørsholm (DK)

(72) Inventors: Knud Jørgen JENSEN, Copenhagen Ø (DK); Charlotte Stahl MADSEN, Måløv (DK); Karin Margareta Sophia MANNERSTEDT, Copenhagen Ø (DK); Esben Matzen BECH, Copenhagen V (DK); Søren Ljungberg PEDERSEN, Borup (DK); Jacob JELSING, Roskilde (DK); Niels VRANG, Klampenborg (DK)

(73) Assignee: GUBRA APS, Hørsholm (DK)

(21) Appl. No.: 16/470,746

(22) PCT Filed: Dec. 22, 2017
(86) PCT No.: PCT/EP2017/084425

§ 371 Date: Jun. 18, 2019

(30) Foreign Application Priority Data
Dec. 22, 2016 (EP) 16206211.1
Oct. 4, 2017 (DK) PA 2017 70754

(51) Int. Cl. A61K 47/64; A61K 38/28; A61K 47/54; A61P 3/10

(52) U.S. Cl. CPC A61K 47/64; A61P 3/10; A61K 47/542; A61K 38/28

(57) ABSTRACT

The present invention relates to a conjugate of the formula P-L-I, wherein P is a peptide hormone effecting the metabolism of carbohydrates in vivo, L is a hydrolysable linker molecule consisting of Lₚ and Lᵢ, and I is a molecule capable of inhibiting the effect of the peptide hormone P on the metabolism of carbohydrates in vivo. Under in vivo conditions, the conjugate is the major compound. When the concentration of glucose increases in vivo, the concentration of the peptide hormone effecting the metabolism of carbohydrates in vivo also increases.

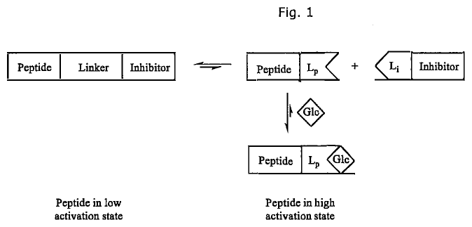

### FIELD OF THE INVENTION

The present invention relates to glucose-responsive peptide conjugates comprising a peptide hormone affecting the metabolism of carbohydrates in vivo, and an agent inactivating or inhibiting the activity of the peptide hormone (or an agent facilitating inactivation or inhibition of the activity of the peptide hormone) conjugated via a hydrolysable linker molecule.

Further, the present invention relates to the use of the glucose-responsive peptide conjugates as a medicament, in particular for use as a medicament in the treatment of diabetes.

The peptide hormone part of the conjugates according to the present invention is in an inactive state in the conjugate due to the presence of the inhibitor inactivating or inhibiting the activity of the peptide hormone.

The hydrolysable linker of the conjugate facilitates the existence of the peptide hormone, the inhibitor and the conjugate in a dynamic equilibrium in vivo.

The present invention further relates to pharmaceutical or veterinary compositions comprising a conjugate according to the invention and at least one pharmaceutical or veterinary excipient.

In the presence of a carbohydrate, such as glucose, the peptide hormone part of the conjugate is removed from the equilibrium when bound to the carbohydrate (although the peptide hormone-bound carbohydrate participates in a new dynamic equilibrium between the peptide hormone, carbohydrate and peptide hormone-carbohydrate conjugate), whereby the pool of non-conjugated peptide hormone parts is increased and the hormone activity increases such that the concentration of the active peptide hormone parts increases in response to increasing concentrations of glucose in vivo. Alternatively, in the presence of a carbohydrate, such as glucose, glucose facilitates a shift in the equilibrium giving more active P.

### BACKGROUND OF THE INVENTION

Peptides, in particular hormones, are frequently used as therapeutic agents to cure or manage a range of diseases. A range of therapeutic peptide hormones that have a therapeutic effect on the metabolism of carbohydrates are used in the management of a range of diseases in humans, such as diabetes, obesity and metabolic disorders.

Preferably, the activity of these peptides is needed in response to rising levels of blood glucose (i.e. rising glucose concentrations in vivo), and therefore a range of the therapeutic peptides affecting the metabolism of carbohydrates are to be administered after a meal, i.e. in response to rising blood glucose levels.

Such administration is cumbersome and requires frequent administrations as well as constant monitoring of the patient.

These problems could be solved by administering the peptide hormones as a depot that releases and/or activates the active peptide hormones in response to rising glucose concentrations in vivo.

Several technologies for achieving polypeptides with increased stability and efficacy through covalent linkage to stabilising molecules exist. Such polypeptides differ fundamentally from the glucose-responsive peptide conjugates according to the present invention in that the polypeptide conjugates according to the present invention are hydrolysable under conditions resembling conditions in vivo in the human body.

As an example, WO 2009/067636 A2 describes in example 12 an insulin polypeptide conjugate comprising the insulin polypeptide conjugated to PEG via a hydrazine linkage that has been reduced in situ to a stable hydrazine linker. The resulting polypeptide is stable and the hydrazine linkage cannot be hydrolysed in vivo. Insulin conjugates according to WO 2009/067636 A2 thereby differ fundamentally from the glucose-responsive peptide conjugates according to the present invention.

WO 2009/059278 A1 describes polypeptides with increased stability due to linkage to Fc-molecules. In claim 7 of this reference, a method of preparing such molecules is described. In performing that method, an intermediate hydrazone comprising an activated GLP-1 peptide and an Fc molecule are formed, which are subsequently reduced to the final stable hydrazine product.

J. Mu et al. (“FGF21 Analogs of sustained Action Enabled by Orthogonal Biosynthesis Demonstrate Enhanced Antidiabetic Pharmacology in Rodents”, Diabetes, Vol. 61, no. 2, 30 Dec. 2011) describes FGF21 stabilised via an oxime adduct to PEG. The stable peptide conjugates according to Mu et al., thereby differ fundamentally from the glucose-responsive peptide conjugates according to the present invention.

In contrast to the above disclosures, the present invention relates to a peptide hormone effecting the metabolism of carbohydrates in vivo, wherein the peptide hormone is conjugated to an inactivating moiety via a hydrolysable linker molecule, whereby an equilibrium between the inactivated peptide hormone and the active peptide hormone is created in vivo. Thereby, e.g. a glucose-dependent insulin activity can be achieved in vivo.

Several technologies for achieving glucose-dependent release of insulin are known.

For example, insulins conjugated to phenylboronic acids (PBA) can bind D-glucose through the PBA moiety. Hoeg-Jensen et al. have described such glucose-sensing insulins (Hoeg-Jensen et al., J. Pept. Sci. 2005, 11, 339-346). Boronate-insulins formulated in for example D-glucosamine polyamide polymers enable a release of insulin in the presence of glucose by displacement. Moreover, Chou et al. have reported phenylboronic acid-lipidated insulins that bind to plasma proteins, such as HSA, and in the presence of glucose, the boronic acid-insulin HSA complex will be disrupted, thus increasing the free insulin fraction in the blood (Chou et al. PNAS, 2014, 112 (8), 2401-2406). A challenge with the PBA technology is the lack of specificity and the high affinity to other diols such as fructose.

ConA (Concanavalin A) is a lectin that binds glucose and maltose. If formulated in a polymer, ConA can bind glucose during hyperglycaemic conditions leading to a swelling or breakdown of the polymer and a release of insulin (Brownlee et al., Science, 1979, 206 (4423), 1190-1191; Zion T C., 2004, PhD thesis Massachusetts Institute of Technology, “Glucose-responsive materials for self-regulated insulin delivery”). A challenge with this method is the immunological responses to non-native ConA molecules and the stability of the ConA native molecules.

Glucose oxidase is highly specific for glucose and transforms glucose to oxygen, hydrogen peroxide, and gluconic acid. Formulating glucose oxidase in microgels or nanoparticles in the body will result in an acidic microenvironment during hyperglycaemic conditions, which leads to an insulin release (Gu et al., ACS Nano, 2013, 7 (8), 6758-6766; Luo et al, Biomaterials, 2012, 33, 8733-8742; Qi et al., Biomaterials, 2009, 30, 2799-2806). A challenge with the latter method is that it is cytotoxic, as hydrogen peroxide has to be quenched in the sensor. The technology has slow response rates and is susceptible to pH.

Consequently, there is a ubiquitous need in the art for new means and methods for providing peptide hormones to obtain altered, preferably increased, activity in response to rising glucose concentrations in vivo.

Accordingly, one object of the present invention is to provide means and methods for altering, preferably increasing, the activity of a peptide hormone in response to rising glucose concentrations in vivo in the human or animal body.

A further object of the present invention is to provide means and methods for altering, preferably decreasing, the activity of a peptide hormone in response to falling glucose concentrations in vivo in the human or animal body.

A further object of the present invention is to provide means and methods for altering the activity of a peptide hormone in response to fluctuating glucose concentrations in vivo in the human or animal body such that the activity of the peptide hormone decreases in response to falling glucose concentrations and increases in response to increasing concentrations of glucose in vivo in the human or animal body.

Further, an object of the present invention is to provide glucose-responsive therapeutic peptide conjugates.

### Definitions

According to the present invention, peptide conjugates are conjugates comprising a first part comprising a peptide hormone and a second part comprising an inactivating means, i.e. a means for inactivating the peptide hormone herein also referred to as an “inhibitor”, the first and the second part being conjugated via a hydrolysable linker moiety.

According to the present invention, peptide hormones are peptides that activate or inactivate certain molecular pathways in vivo, whereby the metabolic activity of a subject to which the peptide hormone is administered is altered. Preferably, peptide hormones according to the present invention include pancreatic hormones, such as insulin or amylin, gut hormone such as glucagon-like peptide-1 (GLP-1), gastric inhibitory polypeptide (GIP, also known as glucose-dependent insulinotropic peptide) or cholecystokinin (CCK), adipocyte-derived hormone such as adiponectin or leptin, myokines such as interleukin 6 (IL-6) or interleukin 8 (IL-8), liver-derived hormone such as betatrophin, fibroblast growth factor 19 (FGF19) and fibroblast growth factor 21 (FGF21). Further, the peptide hormones according to the present invention may be brain-derived proteins such as brain-derived neurotrophic factor (BDNF) and growth hormones.

According to the present invention, an insulin analogue is a peptide having an insulin-like function in vivo in the human or animal body, i.e. a function in the regulation of the metabolism of carbohydrates, fats and proteins by promoting the absorption of especially, glucose from the blood into fat, liver and skeletal muscle cells.

According to the present invention, hydrolysable linker means compounds that bind the peptide hormone and the inhibitor together, but that are prone to a certain extent of hydrolysis under in vivo conditions such that the majority of the peptide hormone parts of the conjugates is present in association with the inhibitor, i.e. as parts of the peptide conjugates according to the invention, under in vivo conditions (at normal blood glucose levels), and a minority of the peptide hormone parts of the conjugates is present free of the linker compounds under in vivo conditions (at normal blood glucose levels). According to the present invention, a linker is hydrolysable in vivo if the linker hydrolyses in vitro in phosphate buffer pH 7.4 such that an equilibrium between linker and hydrolysed linker exists within 5 hours such that at least 1% and up to 50% of the linker is hydrolysed.

According to the present invention, at least one of the conjugate parts P-Lₚ and Lᵢ-I binds covalently to glucose in vivo, if the linker, under conditions as described in example 6, produce a conjugate between glucose and at least part of the linker within 96 hours, preferably within 72 hours, more preferably within 24 hours.

According to the present invention the hydrolysis of the hydrolysable linker L is being promoted by glucose if the linker hydrolyses in vitro in phosphate buffer pH 7.4 in the presence of 10.000 equiv. glucose, such that an equilibrium between linker and hydrolysed linker exist within 5 hours such that at least 2% and up to 100% of the linker is hydrolysed and such that the amount of hydrolysed linker is increased by the presence of glucose.

According to the present invention, the inactivator (I) is a molecule capable of inactivating the active site of a peptide (P). Such molecule (I) may be e.g. a molecule capable of limiting the exposure of the active site of P to the environment. As an example, limiting the exposure of the active site of P to the environment may e.g. be achieved by binding P (via the hydrolysable linker and the inactivator part of the conjugate) to macromolecular substances such as PEG, Fc antibody, XTEN, PASylation, serum albumin (covalent), carbohydrate polymers (such as dextran, HES, polysialylation), nanoparticles and hydrogels. According to the present invention, the inactivator (I) is a molecule capable of inactivating a peptide (P), if, under conditions as described in example 9 (where P is insulin), the activity of PI is 50% or less of the activity of P.

According to the invention, the inhibitor or inactivator (I) may alternatively be a molecule capable of non-covalent binding to larger protein structures in human serum, thereby facilitating the clustering of multiple conjugates according to the invention in vivo. According to this aspect of the invention, the inhibitor or inactivator (I) may be a small molecule albumin binder or a lipid molecule, or any molecule capable of non-covalent binding to serum albumin.

An inactivator or inhibitor of insulin may also be a molecule that is bound, linked via L, to insulin at a position that inhibits the activity of insulin.

According to the present invention, a molecule capable of inactivating the active site of a peptide (P), is a molecular structure which, when present in the conjugate, is responsible for decreasing the activity of the relevant peptide hormone to an extent that the activity of the relevant peptide hormone is reduced to less than 50%, preferably less than 40%, even more preferably less than 30%, even more preferably less than 20%, and most preferably to less than 10% of the activity of the peptide (P) (i.e. the activity of P in the absence of the molecule capable of inactivating the active site of a peptide (P)) under in vitro conditions as described in example 9 (where P is insulin). The inhibition capability of the inactivator or inhibitor may be measured using a functional receptor assay for the peptide “P”. First, the functionality (EC50) of the P-L-I molecule dissolved in PBS, pH 7.4, could be measured, and secondly, the P-Lₚ-Glc or P-L molecule could be measured (if relevant in the presence of a relevant macromolecular structure). P-Lₚ-Glc could be formed by adding 1.000 equivalents glucose to a P-L-I mixture, dissolved in PBS pH 7.4, and left to react for 72 h. If the inhibitor “I” is a molecule capable of non-covalent binding to larger protein structures in human serum, such as a molecule capable of binding to a plasma protein, the relevant structure or protein should be included in the experiment. The functionality (EC50) of P-L-I compared to P-Lₚ-Glc determines the inhibitor “I” ability to decrease the activity of the peptide “P”.

### SUMMARY OF THE INVENTION

The peptide conjugates according to the present invention address and solve the problem of altering the hormonal activity of a peptide hormone in response to fluctuating carbohydrate concentrations in vivo by creating a dynamic equilibrium releasing active peptide hormones in response to rising glucose concentrations in vivo. In response to falling glucose concentrations in vivo, the pool of active peptide hormones is decreased due to less release from the pool of conjugated peptides, and a relatively short half-life of the peptide hormone itself.

The invention thus provides new methods and means for providing glucose-responsive therapy. The therapeutic peptide conjugates according to the invention are glucose-responsive by consisting of a first part comprising an active peptide hormone, which is coupled to a second part comprising an inactivating means. The inactivation means may inactivate the peptide hormone by e.g. facilitating depot formation, facilitating binding to large molecules, such as serum albumin, or by directly inhibiting the active site of the peptide hormone. Conjugates consisting of a first part comprising a peptide hormone coupled to a second part comprising means that inactivate the peptide hormone are known in the art, i.e. as insulin depots wherein insulin is covalently or non-covalently coupled to larger molecules such as serum albumin. These insulin depots slowly and constantly deliver insulin to the body in vivo.

The present invention resides e.g. in the use of a hydrolysable linker to associate the peptide hormone and the inactivating means, where the hydrolysable linker (or a part thereof) is capable of binding a carbohydrate, preferably glucose, after hydrolysis. Re-association after hydrolysis is prevented by the presence of a carbohydrate, preferably glucose. In an alternative embodiment, the presence of the carbohydrate, preferably glucose, prevents the reformation of the linker (L) after the hydrolysis of L through another mechanism. In an alternative embodiment, the presence of the carbohydrate, preferably glucose, promotes the hydrolysis of L.

The first and the second parts of the conjugate of the invention are linked via a hydrolysable linker. At least one part of the hydrolysable linker binds glucose after being hydrolysed, or, alternatively glucose promotes the hydrolysis of the hydrolysable linker.

In solution, e.g. in vivo, the conjugates according to the invention will be present in a dynamic equilibrium comprising the inactive peptide conjugate (where the linker is unhydrolysed) as well as the two parts thereof in isolation, i.e. the active peptide hormone, where the linker is hydrolysed, and the inactivation means in isolation.

When glucose is present, glucose will bind to at least one part of the hydrolysable linker, whereby the glucose-bound part, i.e. the glucose-bound active peptide hormone and/or the glucose-bound inactivating means, will no longer take part in the dynamic equilibrium between PLI, PL_{P} and L_{I}I.

The dynamic equilibrium will replace the removed parts and thereby deliver new active peptide hormones when the glucose concentration increases.

In an alternative embodiment, when glucose is present, glucose promotes the hydrolysis of the hydrolysable linker, whereby the dynamic equilibrium is altered such that an increased amount of active peptide hormone is formed in the dynamic equilibrium.

### DETAILED DESCRIPTION OF THE INVENTION

The present invention is based on the inventive finding that peptide hormones, and the activity thereof, can be made responsive to glucose concentrations in vivo by coupling the peptide hormones to an inactivating means via a hydrolysable linker that binds glucose when hydrolysed, or the hydrolysis of which is promoted by glucose.

Thereby, a dynamic equilibrium exists in vivo between the active peptide hormone and the inactivated peptide conjugate.

In the absence of glucose (Glc), or in the presence of very low concentrations of glucose, the majority of the peptide hormones will be in the form of peptide conjugates according to the invention, i.e. they will be in the inactivated form due to the dynamic equilibrium favouring the inactivated conjugate.

However, in the presence of glucose, glucose binds to the active peptide hormone and/or the inactivation agent, whereby the formation of inactivated peptide conjugate from that peptide hormone, to which glucose is bound, is hindered. In such a situation, the dynamic equilibrium will produce one active peptide hormone from the reservoir of inactive peptide conjugates for each peptide hormone being associated with glucose. In other words, the presence of glucose will initiate the release of active peptide hormones from the reservoir of peptide hormones being present as part of an inactive peptide conjugate. Falling concentrations of glucose will initiate decreased levels of active peptide hormones. As another alternative, the same effect may be achieved by the presence of the carbohydrate, preferably glucose, preventing the reformation of the linker (L) after the hydrolysis of L by any other mechanism.

Alternatively, the same effect may be achieved by the hydrolysis of the hydrolysable linker being promoted by glucose.

This finding paves the way for e.g. producing glucose-responsive depots of peptide hormones, such as glucose-responsive depots of insulin.

Accordingly, in its broadest aspect, the present invention relates to a conjugate of the formula P-L-I, wherein P is a peptide hormone effecting the metabolism of carbohydrates in vivo, L is a linker molecule consisting of Lₚ and Lᵢ-, and I is a molecule capable of inactivating or inhibiting the effect of the peptide hormone P on the metabolism of carbohydrates in vivo, characterised in that:
a. the linker molecule L is hydrolysable in vivo such that the conjugate P-L-I and the hydrolysed conjugate parts P-Lₚ+Lᵢ-I exist in a dynamic equilibrium in vivo where the conjugate P-L-I exists in molar excess of at least one of the conjugate parts P-Lₚ and Lᵢ-I, and further characterised in that
b. at least one of the conjugate parts P-Lₚ and Lᵢ-I binds covalently to glucose whereby the concentration of P that is not conjugated to I increases in vivo when the concentration of glucose increases in vivo, or, alternatively further characterised in that the hydrolysis of the hydrolysable linker L is being promoted by glucose.

P:

P is a peptide hormone effecting the metabolism of carbohydrates in vivo.

In one aspect of the invention, the peptide hormone P is a pancreatic hormone, such as insulin or amylin. In another aspect of the invention, the peptide hormone is a gut hormone, such as glucagon-like peptide-1 (GLP-1), gastric inhibitory polypeptide (GIP, also known as the glucose-dependent insulinotropic peptide) or cholecystokinin (CCK) or analogues thereof. In another aspect of the invention, the peptide hormone is an adipocyte-derived hormone such as adiponectin or leptin. In another aspect of the invention, the peptide hormone is a myokine such as interleukin 6 (IL-6) or interleukin 8 (IL-8) or analogues thereof. In another aspect of the invention, the peptide hormone is a liver-derived hormone such as betatrophin, fibroblast growth factor 19 (FGF19) or fibroblast growth factor 21 (FGF21) or analogues thereof. In another aspect of the invention, the peptide hormone is a brain-derived protein, such as brain-derived neurotrophic factor (BDNF) or analogues thereof. In another aspect of the invention, the peptide hormone is a growth hormone or an analogue thereof.

In a highly preferred aspect of the invention, the peptide hormone is insulin or an analogue thereof, or a molecule capable of activating the insulin receptor (INR).

According to the present invention, peptide hormones to which glucose is bound are also comprised by the definition of P.

I:

I is a molecule or substance that is capable of inactivating or inhibiting the effect of the peptide hormone P on the metabolism of carbohydrates in vivo e.g. by facilitating inactivation or inhibition of the activity of the peptide hormone by formation of inactive complexes in vivo or by direct inhibition of the active site of the peptide hormone.

Molecules and mechanisms capable of inactivating or inhibiting the effect of the peptide hormone P on the metabolism of carbohydrates in vivo are well-known in the art. Inactivating or inhibiting a peptide hormone in vivo may, in general, be achieved by limiting the exposure of the active site of P to the environment. As an example, limiting exposure of the active site of P to the environment may e.g. be achieved by binding P (via the hydrolysable linker and the inactivator part of the conjugate) to macromolecular substances such as PEG, Fc antibody, XTEN, PASylation, serum albumin (covalent), carbohydrate polymers (such as dextran, HES, polysialylation), nanoparticles and hydrogels. Alternatively, I may be small molecule albumin binders or lipids capable of non-covalent binding to serum albumin. An inactivator or inhibitor of insulin may also be a molecule that is bound, linked via L, to insulin at a position that inhibits the activity of insulin.

When present in the conjugate, an inactivator or inhibitor according to the present invention should be responsible for decreasing the activity of the relevant peptide hormone to an extent that the activity of the relevant peptide hormone is reduced to less than 50%, preferably less than 40%, even more preferably less than 30%, even more preferably less than 20%, and most preferably to less than 10% of the activity in the absence of the attached inactivator or inhibitor under in vitro conditions. The inhibition capability of the inactivator or inhibitor may be measured using a functional receptor assay for the peptide “P”. First, the functionality (EC50) of the P-L-I molecule dissolved in PBS, pH 7.4, could be measured, and secondly, the P-Lₚ-Glc molecule could be measured (if relevant in the presence of a relevant macromolecular structure). P-Lₚ-Glc could be formed by adding of 1.000 equivalents glucose to a P-L-I mixture, dissolved in PBS pH 7.4, and left to react for 72 h. If the inhibitor “I” is a molecule capable of binding to a plasma protein, the protein should be included in the experiment. The functionality (EC50) of P-L-I compared to P-Lₚ-Glc determines the inhibitor “I” ability to decrease the activity of the peptide “P”.

L:

L is a hydrolysable linker molecule consisting of Lₚ and Lᵢ. When L is hydrolysed, a molecule of water is added to L, which results in the fragmentation of L into Lₚ and Lᵢ.

L must be hydrolysable in vitro and in vivo, but preferably L is only hydrolysed at a low frequency, such that L, Lₚ and Lᵢ, exist in a dynamic equilibrium in water under in vitro and in vivo conditions, wherein L (the conjugate) is the major compound and Lᵢ and Lₚ (the conjugate parts) are the minor compounds. In other words, L exists in molar excess of Lₚ and Lᵢ under in vitro conditions, meaning that P-L-I exists in molar excess of P-Lₚ and Lᵢ-I under in vitro conditions. A linker is said to be hydrolysable according to the present invention if it results in the existence of a dynamic equilibrium under in vitro conditions as described in example 6, in which P-L-I exists in molar excess of at least 2:1, preferably at least 3:1, more preferably at least 4:1, even more preferably at least 5:1, even more preferably at least 10:1, even more preferably at least 50:1, and most preferably at least 100:1, with regard to the presence of the conjugate parts P-Lᵢ and/or Lₚ-I. The in vitro hydrolysability of a P-L-I molecule could be measured by dissolving the P-L-I molecule in PBS pH 7.4 and after 24 h, investigate the ratio between P-Lₚ or Lᵢ-I and P-L-I using UPLC-MS.

In a preferred embodiment of the invention, either Lₚ or Lᵢ (or both Lₚ and Lᵢ) must be capable of binding covalently to carbohydrates, such as preferably glucose. After binding to glucose, the respective fragments to which glucose is bound (P-Lₚ-Glc and/or Glc-Lᵢ-I) cannot any longer participate in the formation of the conjugate P-L-I. A compound is said to be able to bind covalently to glucose if it is capable of forming a glucose-conjugated structure within 72 hours of contacting the compound with a molar excess of glucose. The glucose binding capability of a linker in vitro can be measured as shown in example 2. The linker “L” is dissolved in PBS, pH 7.4 together with a 1000 eq. of glucose, and the generated Lₚ-Glc is measured after 24, 48 and 72 h by LC-MS.

In an alternative embodiment, glucose may prevent the re-association of Lₚ and Lᵢ through another mechanism than binding to one or both of Lₚ and Lᵢ.

In yet another alternative embodiment, glucose promotes, facilitates or enhances the hydrolysis of L.

In a preferred aspect of the invention, L is either a hydrazone, O,O-acetal, N,O-acetal, N,N-acetal, S,N-acetal including thiazolidine and thiazoline, or S,S-acetal including dithiolane, and their derivatives.

Hydrazones are especially preferred due to their well-described chemistry, ease of formation, and the straight-forward possibility to tune the stability and lability of the bond towards hydrolysis and other reactions.

Although acetals (including with O, N, S) are expected to exchange slower than hydrazones, acetals are also especially preferred due to the possibility to tune the stability and lability of the bond towards hydrolysis and other reactions, as well as the formation of cleavage products that are readily biologically degraded.

In a highly preferred aspect of the invention, L is a hydrazone of the general formula 1:

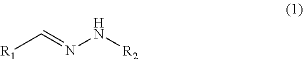

wherein, R₁ is preferably an aromatic ring with a 1-10 carbon spacer alkyl chain between the aromatic ring and the hydrazone, and

R₂ is preferably a benzoyl.

Preferably, R₁ is an aromatic ring with weak to moderately activating (electron donating) or deactivating (electron withdrawing) substituents attached to the hydrazone via an alkyl linker.

Most preferably, R₂ is a benzoyl with moderate to strongly electron donating substituent(s) such as -amide, —OMe, —N(CH₃)₂ or —OH.

In particular, L may be a conjugate of the general formula 2:

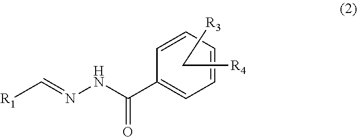

wherein,

R₁ is preferably an aromatic ring with a 1-10 carbon spacer alkyl chain between the aromatic ring and the hydrazone, and

R₃ is an electron donating group, and

R₄ comprises P or I.

Most preferably, R₁ is an aromatic ring with weak to moderately activating or deactivating substituents attached to the hydrazone via an alkyl linker.

In a preferred embodiment of the invention, L is a conjugate of the general formula

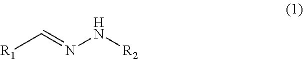

wherein R₁ is selected among:

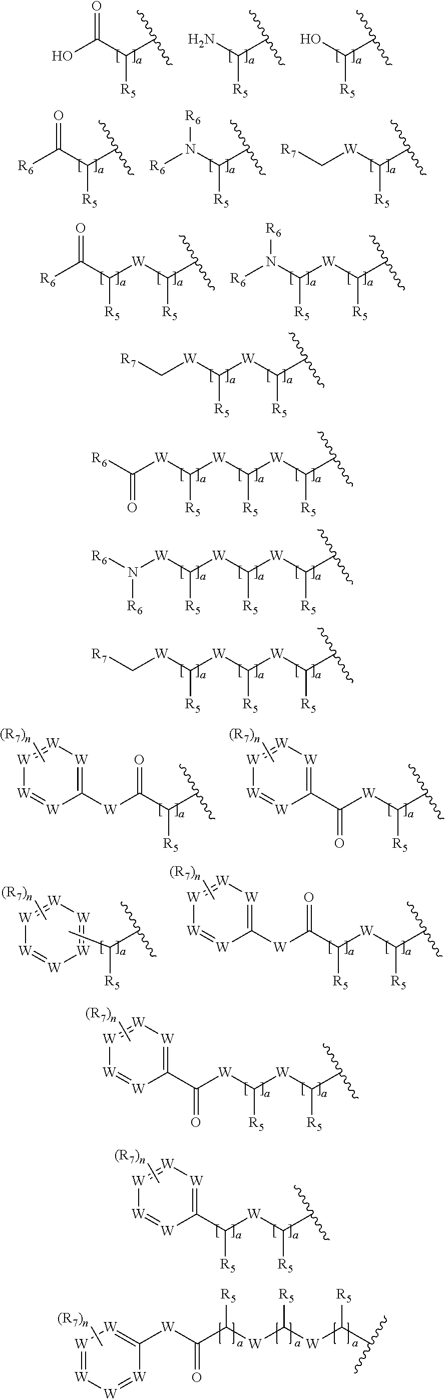

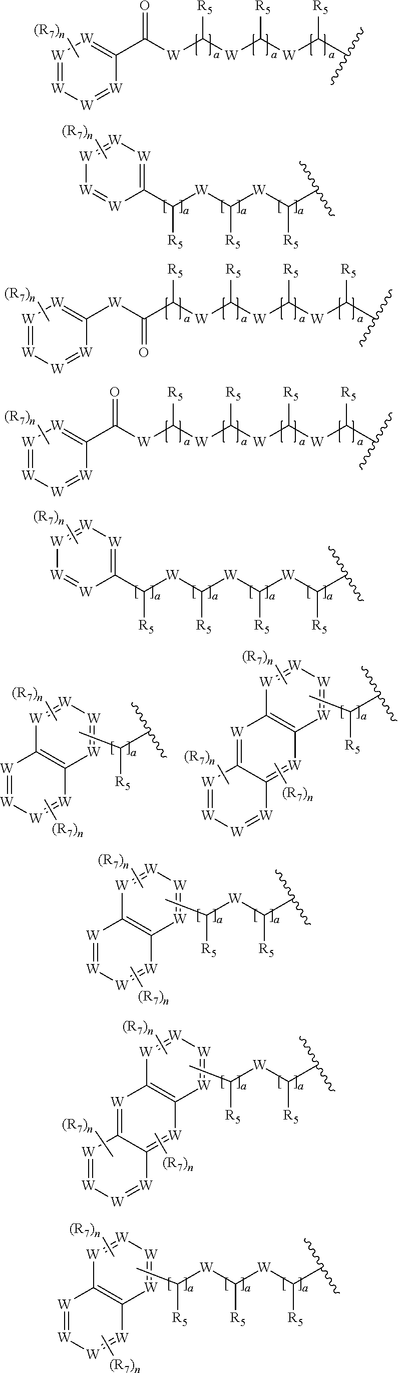

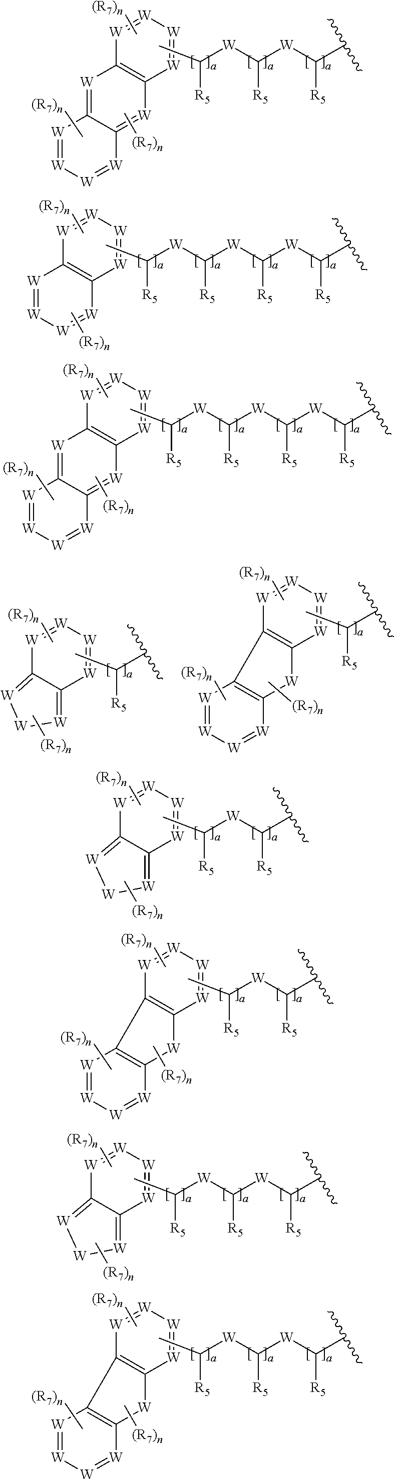

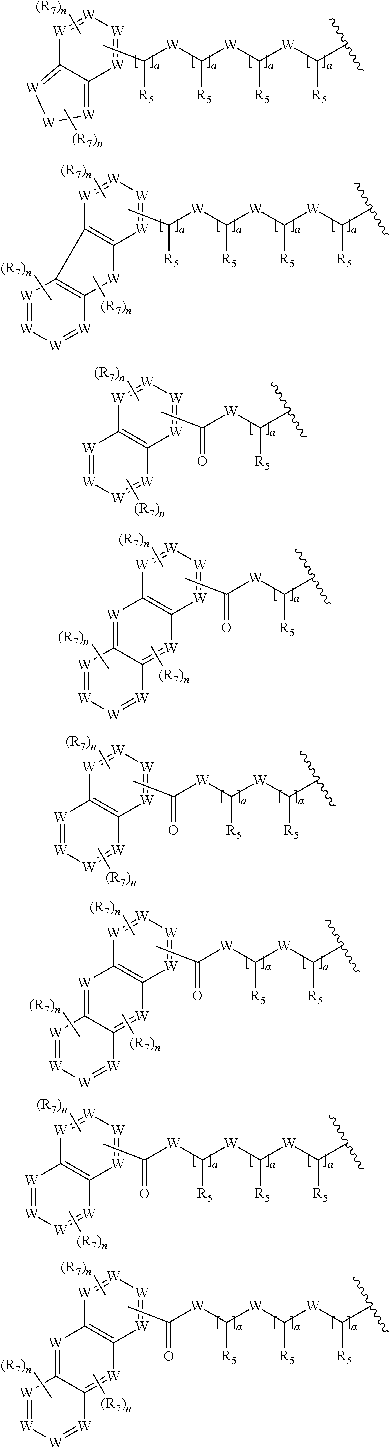

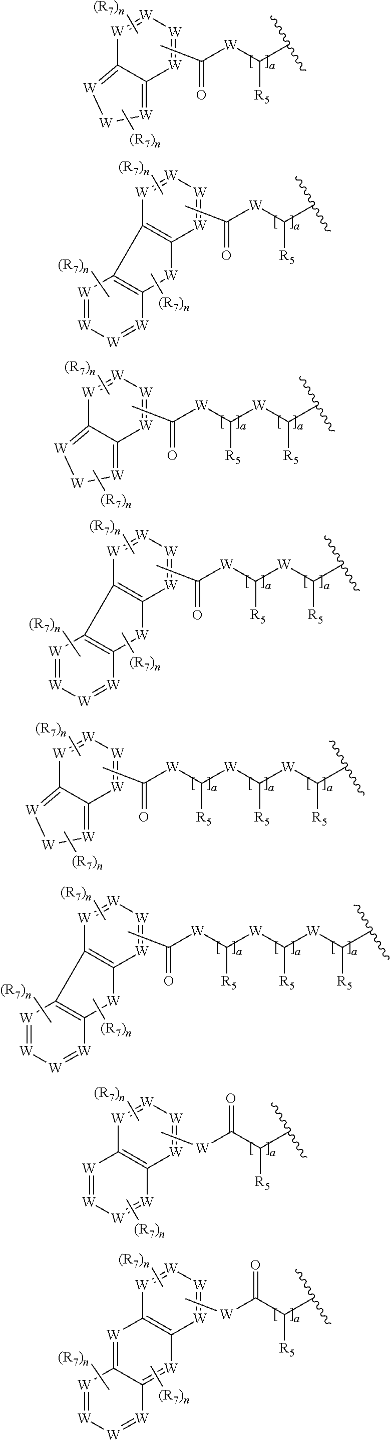

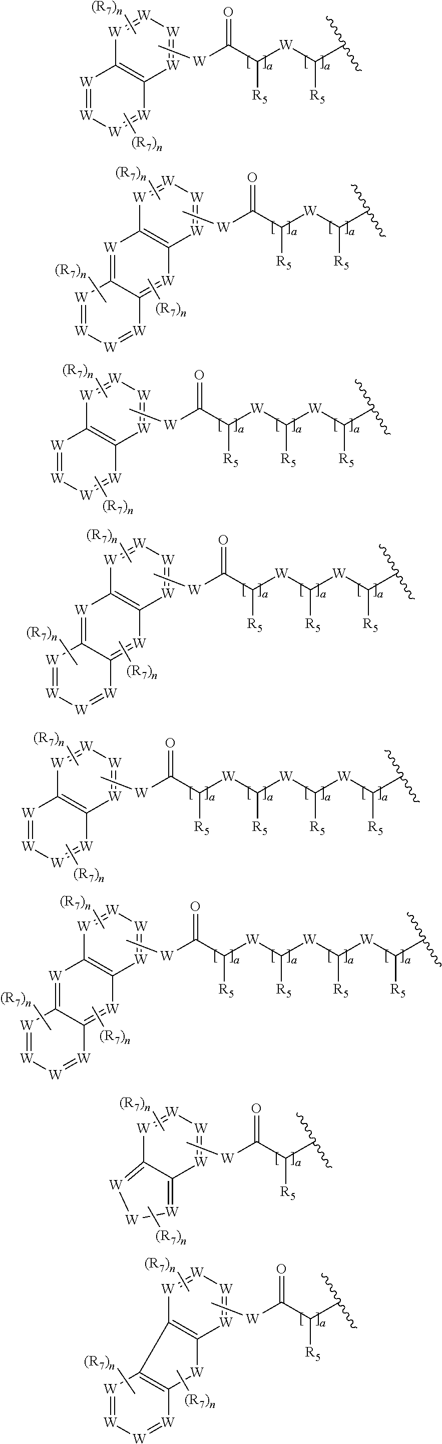

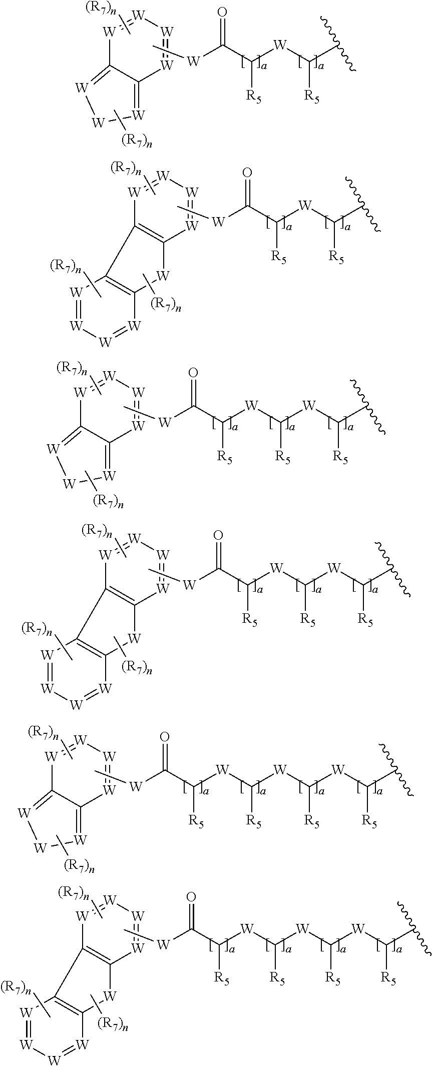

where a is 1-10; n is 0-4; R₅ is hydrogen, methyl or ethyl; R₆ is hydrogen, methyl, ethyl, an alkane, the peptide (P) and/or the inhibitor (I); R₇ is hydrogen, O-benzyl, O-methyl, O-alkane, amide, amine, halogen, NO₂, the peptide (P) and/or the inhibitor (I); W is carbon (CH₂, CH or C), nitrogen (NH), NCH₃, sulfur (S) and/or oxygen (O),

and where R₂ is selected among:

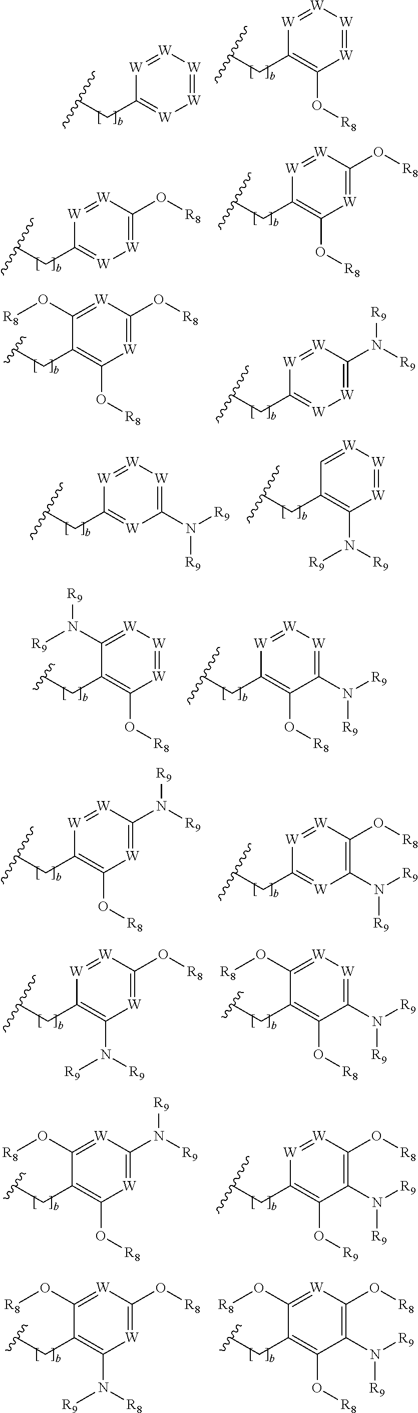

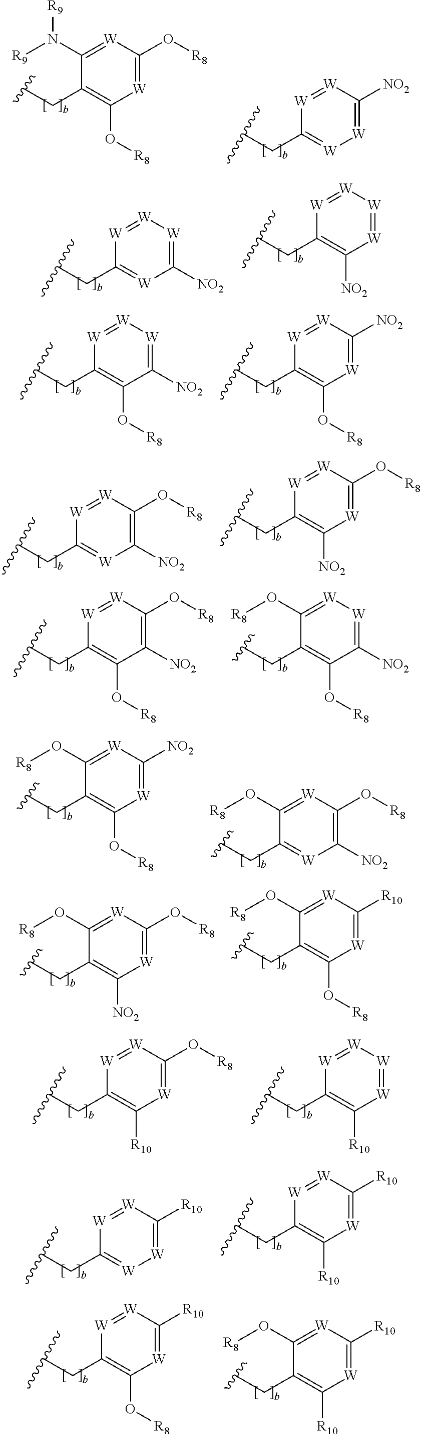

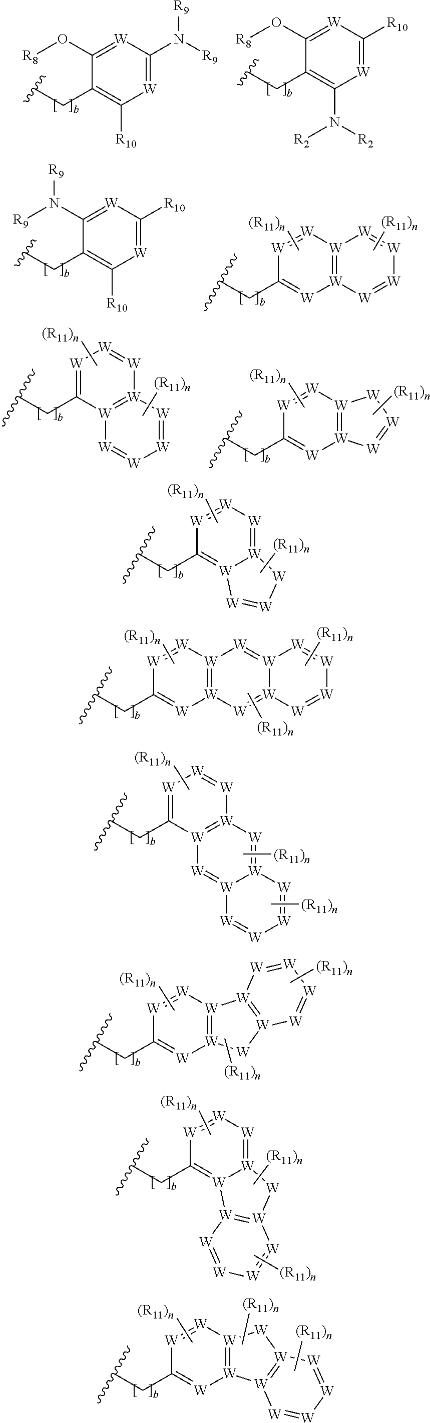

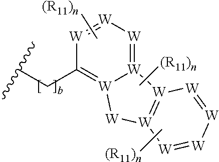

where b is 1-10; n is 0-4, R₈ is hydrogen, benzyl, methyl, alkane, the peptide (P) and/or the inhibitor (I); R₉ is hydrogen, methyl, alkane, the peptide (P) and/or the inhibitor (I); R₁₀ is halogen (Cl, Br, I or F), an ester, carboxylic acid and/or the inhibitor I; R₁₁ is hydrogen, O-benzyl, O-methyl, O-alkane, amide, amine, halogen, the peptide (P) and/or the inhibitor (I), and W is carbon (CH₂, CH or C), nitrogen (N or NH), sulfur (S) and/or oxygen (O).

P-L-I:

The conjugate of the formula P-L-I according to the invention is a conjugate comprising the above-mentioned components P, L and I.

Due to the hydrolysable nature of L, the conjugate P-L-I exists in vivo in a dynamic equilibrium

wherein P-L-I is in molar excess of one or both of P-Lₚ and Lᵢ-I.

Due to the association of P and I, the conjugate P-L-I is inactive (or has a reduced efficacy) in vivo, whereas the peptide hormone P-Lₚ, as well as the peptide hormone P-Lₚ-Glc, is an active peptide hormone in vivo.

In a highly preferred aspect of the invention, P-Lₚ binds covalently to glucose (Glc).

Thereby, activated P (P that is no longer associated with I) is then blocked from further associating with the inhibitor. As an example, if L is a hydrazone, P-Lₚ is a hydrazide. The hydrazide may react with glucose to form a new hydrazone, P-L_{P}-Glc. Thereby, the hydrazide of the active peptide hormone is blocked from reacting further with the inhibitor, by binding to glucose. In theory, the P-Lₚ-Glc molecule is in a new equilibrium with P-Lₚ and glucose, but as the glucose concentration is more than 10,000 equivalents higher than the P-Lₚ part in vivo, it is anticipated that when glucose has bound to P-Lₚ to form P-Lₚ-Glc, the dissociation is very slow and thus, P-Lₚ-Glc can be regarded as a stable molecule. In contrast, the Lᵢ-I part is now an aldehyde, which may react with other components.

In an alternative embodiment of the invention, the hydrolysis of the hydrolysable linker L is promoted by glucose, whereby the dynamic equilibrium is altered in the presence of glucose.

In a highly preferred aspect of the invention, P is insulin or an insulin analogue or a molecule capable of activating the insulin receptor (INR). Preferably, P is capable of activating the insulin receptor below μM concentrations, such as at a concentration of less than 1 μM.

In a further aspect, the present invention relates to the use of a conjugate of the formula P-L-I for the treatment of a disease in a human being, wherein P is a peptide hormone effecting the metabolism of carbohydrates in vivo, L is a linker molecule consisting of Lₚ and Lᵢ, and I is a molecule capable of inhibiting the effect of the peptide hormone P on the metabolism of carbohydrates in vivo, characterised in that
a. the linker molecule L is hydrolysable in vivo, such that the conjugate P-L-I and the hydrolysed conjugates P-Lₚ+Lᵢ-I exist in a dynamic equilibrium in vivo where the conjugate P-L-I exists in molar excess of at least one of the conjugate parts P-Lₚ and Lᵢ-I, and further characterised in that
b. at least one of the conjugate parts P-Lₚ and Lᵢ-I binds covalently to glucose, whereby the concentration of P-Lₚ that is not bound to I increases in vivo when the concentration of glucose increases in vivo, or, alternatively further characterised in that the hydrolysis of the hydrolysable linker L is being promoted by glucose.

In a further aspect, the present invention relates to a method of treatment of a disease in a subject, the method comprising administering to the subject a conjugate of the formula P-L-I, wherein P is a peptide hormone effecting the metabolism of carbohydrates, preferably glucose, in vivo, L is a linker molecule consisting of Lₚ and Lᵢ, and I is a molecule capable of inhibiting the effect of the peptide hormone P on the metabolism of carbohydrates in vivo, characterised in that
a. the linker molecule L is hydrolysable in vivo, such that the conjugate P-L-I and the hydrolysed conjugate parts P-Lₚ+Lᵢ-I exist in a dynamic equilibrium in vivo where the conjugate P-L-I exists in molar excess of at least one of the conjugate parts P-Lₚ and Lᵢ-I, and further characterised in that
b. at least one of the conjugate parts P-Lₚ and Lᵢ-I binds covalently to glucose, whereby the concentration of P-Lₚ that is not bound to I increases in vivo when the concentration of glucose increases in vivo, or, alternatively further characterised in that the hydrolysis of the hydrolysable linker L is being promoted by glucose.

In a highly preferred aspect of the invention, P is insulin or an insulin analogue.

In a highly preferred aspect of the invention, I is an agent capable of inactivating or inhibiting P by facilitating depot formation, e.g. by facilitating binding to large molecules, such as serum albumin.

In another highly preferred aspect of the invention, I is an agent capable of inhibiting the active site of P, e.g. an inhibitor that is bound to the peptide hormone (e.g. insulin) at a position that inhibits the activity of the peptide hormone.

In another highly preferred aspect of the invention, I is an agent capable of inactivating or inhibiting P by facilitating depot formation, e.g. by facilitating binding of P to large molecules, such as serum albumin. Alternatively, I may be an agent capable of clustering multiple components in structures, such as hydrogels or nanoparticles.

In another aspect of the invention, I is a large molecule, such as serum albumin.

In another aspect of the invention, I is a hydrogel. A hydrogel is a hydrophilic gel that consists of a network of polymer chains in which water is the dispersion medium. In this aspect of the invention, the hydrogel is the inhibitor (I), and chemical handles on the hydrogel allow for covalent attachment of the peptide hormone (P) via the linker (L).

In one aspect of the invention, I is a nanoparticle. Nanoparticles (which may be viewed as a type of colloidal drug delivery system) comprise particles with a size range from 2 to 1000 nm in diameter. In this aspect of the invention, the nanoparticles may be coated with a polymer allowing covalent attachment of the peptide hormone (P) via the linker (L).

However, in a highly preferred aspect, I is an agent capable of non-covalently binding to serum albumin, such as fatty acids or small molecule albumin binders, or other plasma proteins.

In a highly preferred aspect of the invention, I is an agent capable of inactivating or inhibiting P by facilitating depot formation, e.g. by facilitating binding of P to large molecules, such as serum albumin. Preferably, such agent is a fatty acid, which comprises the structure A, where A is selected among;

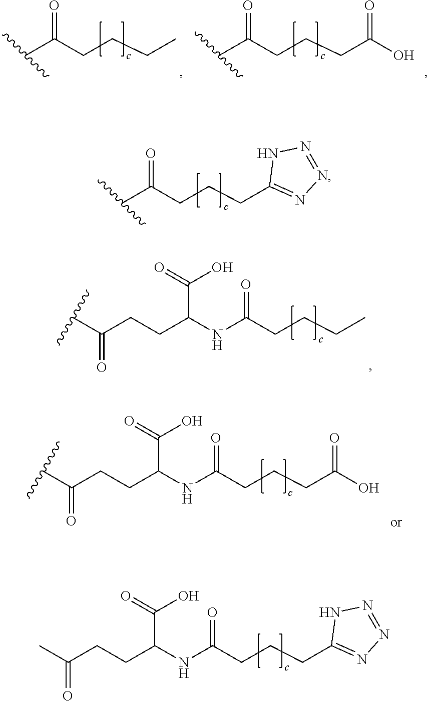

and c is at least 10.

Other Preferred Inhibitors (I):

In another highly preferred aspect, I is a large molecule that prevents the conjugated peptide from being cleared in the kidney. Such molecules may be recombinant albumin, Fc antibody, PEG, or carbohydrate polymers, such as dextran, hydroxyethyl starch (HES) or a polymer of sialic acids (polysialylation).

In addition to inhibiting the activity of the hormone P in the conjugate, recombinant albumins are able to load peptides (P) via the linker (L) leading to low renal excretion of the peptide hormone P, providing a system that is longer lasting in vivo. Similarly, conjugating the peptide (P) via the linker (L) to the Fc part of the IgG antibody enables recycling of the conjugate via the Fc receptor leading to low renal clearance. In the same way, chemical conjugation of the peptide (P) via the linker (L) to polyethylene glycol (PEG), using PEG20 to PEG80, prevents renal excretion by increasing the hydrodynamic volume of the peptide. Accordingly, in one highly preferred aspect of the invention, I is a recombinant albumin, Fc antibody or PEG.

In the same way, carbohydrate polymers, such as dextran, hydroxyethyl starch (HES) or polysialylated conjugates thereof, may prevent the conjugated peptide from being cleared in the kidney. Dextran polymers may be obtained from bacteria such as L. mesenteroides and are D-glucose polymers linked by α(1-6) glycosidic linkages and a small extent of α(1-3) bonds (˜95% α(1-6) and 5% α(1-3) in the case of L. mesenteroides). In addition to unmodified dextran, various synthetic dextran derivatives, such as carboxymethyl-dextran (CMD), diethylaminoethyl dextran (DEAED), glycosylated versions of CMD such as galactose-CMD (Gal-CMD) and mannose-CMD (Man-CMD), carboxymethyl benzylamide dextran (DCMB), carboxymethyl sulfate dextran (DCMSu), and carboxymethyl benzylamide sulfate dextran (DCMBSu) can be used to chemically modify a peptide (P) via the linker (L). Dextran, as PEG, increases the hydrodynamic volume of the peptide leading to a reduced renal filtration. Hydroxyethyl starch (HES) is a modified natural polymer obtained by controlled hydroxyethylation of the plant polysaccharide amylopectin. Amylopectin is a polymer of D-glucose containing primarily α-1,4 glycosidic bonds, but also a lower abundance of α-1,6 linkages, leading to a naturally branched carbohydrate. Hydroxyethylation of the starch precursor serves two purposes: first, to increase the water solubility by increasing the water-binding capacity and decreasing viscosity, and second, to prevent immediate degradation by plasma α-amylase and subsequent renal excretion. HES can be chemically modified in the reducing end allowing for the attachment of the P-L-moiety. ‘Sialic acid’ does not refer to a single chemical entity, but rather to an entire group of nine carbon monosaccharides, the most important examples being 5-N-acetylneuraminic acid (Neu5Ac), 5-N-glycolylneuraminic acid (Neu5Gc), and 2-keto-3-deoxynonulosonic acid (Kdn). However, the only observed polysialic acid (PSA) variant in humans is colominic acid (CA), the linear α-2,8-linked homopolymer of Neu5Ac. Conjugation of polysialic acids to peptides or proteins is referred to as polysialylation. Similar to PEG and the PEG mimetics dextran and HES, the driving force behind the long pharmacokinetic profile of polysialylated conjugates is thought to be an increase in hydrodynamic radius, resulting in decreased renal clearance as well as shielding from enzymatic degradation and antibody recognition.

Preferred Hydrolysable Linkers:

In a preferred aspect of the invention, L is selected among hydrazones, O,O-acetals, N,O-acetals, N,N-acetals, S,N-acetals including thiazolidines and thiazolines, or S,S-acetals including dithiolanes, and their derivatives.

In a highly preferred aspect of the invention, L is a hydrazone or an acetal or a derivative thereof.

In a highly preferred aspect of the invention, L is a hydrazone or a hydrazone derivative.

In particular, L may be a compound of the general formulae

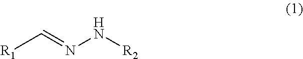

wherein
R₁ comprises I or P, preferably attached to an aromatic moiety, and
R₂ comprises P or I.

In particular, L may be a compound of the general formulae

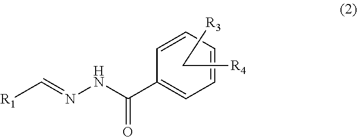

wherein
R₁ comprises an aromatic moiety to which I or P is attached and
R₃ is an electron donating group and
R₄ comprises P or I
R₃ may not be the only electron-donating group of the aromatic moiety.

P or I may also be attached to L via an electron-donating group of the aromatic moiety.

In a highly preferred aspect of the invention, R₁ comprises a spacer region consisting of a carbon chain comprising at least 3 carbon atoms.

The conjugates according to the present invention may be used for the treatment or prophylactic treatment of a human or animal subject.

In particular, the conjugates according to the present invention may be used for the treatment of diabetes mellitus in a human or animal subject. Even more particularly, the conjugates according to the present invention may be used for the treatment of diabetes mellitus in a human or animal subject, the treatment comprising administering the conjugate in a frequency of 2 or less administrations per day. Even more particularly, the conjugates according to the present invention may be used for the treatment of diabetes mellitus in a human or animal subject, the treatment comprising administering the conjugate in a frequency of 1 or less administrations per day.

Thus, the present invention also relates to a method of treatment of diabetes mellitus, said method comprising administering the conjugate according to the invention to a person in need thereof.

In another aspect, the invention relates to a pharmaceutical composition comprising a conjugate according to the invention, and at least one pharmaceutical excipient.

In another aspect, the invention relates to a veterinary composition comprising a conjugate according to the invention and at least one veterinary excipient.

In a highly preferred embodiment, the present invention relates to the use of a conjugate of the formula P-L-I, wherein P is a peptide hormone effecting the metabolism of carbohydrates in vivo, L is a hydrolysable linker molecule consisting of Lₚ and Lᵢ, and I is a molecule capable of inactivating or inhibiting the effect of the peptide hormone P on the metabolism of carbohydrates in vivo, characterised in that
a. the linker molecule L is hydrolysable in vivo, such that the conjugate P-L-I and the conjugate parts P-Lₚ and Lᵢ-I exist in a dynamic equilibrium in vivo where the conjugate P-L-I exists in molar excess of at least one of the conjugate parts P-Lₚ and Lᵢ-I, and further characterised in that
b. at least one of the conjugate parts P-L_{P} and Lᵢ-I binds covalently to glucose, whereby the concentration of P that is not bound to I increases in vivo when the concentration of glucose increases in vivo, or, alternatively further characterised in that the hydrolysis of the hydrolysable linker L is being promoted by glucose,

in the treatment of the human or animal body.

In another highly preferred embodiment, the present invention relates to a conjugate of the formula P-L-I, wherein
P is insulin or an insulin analogue,
L is selected among hydrazones, O,O-acetals, N,O-acetals, N,N-acetals, S,N-acetals including thiazolidines and thiazolines, or S,S-acetals including dithiolanes, and their derivatives, and

I is a molecule capable of non-covalent binding to serum albumin or alternatively, I is serum albumin.

A conjugate of the formula P-L-I wherein I is serum albumin may e.g. be formed in vivo in the human or animal body after administration of P-L.

In a highly preferred embodiment, the present invention relates to the use thereof in the treatment of a human subject. In a highly preferred embodiment, the present invention relates to the use thereof in the treatment of diabetes in a human subject. In a highly preferred embodiment, the present invention relates to the use thereof in the manufacture of a medicament for the treatment of diabetes in a human subject.

In another highly preferred embodiment, the present invention relates to a conjugate of the formula P-L-I, wherein
P is insulin or an insulin analogue,
L is selected among hydrazones, O,O-acetals, N,O-acetals, N,N-acetals, S,N-acetals including thiazolidines and thiazolines, or S,S-acetals including dithiolanes, and their derivatives, and
I is a molecule capable of non-covalent binding to serum albumin or alternatively, I is serum albumin, characterised in that
   a. the linker molecule L is hydrolysable in vivo, such that the conjugate P-L-I and the conjugate parts P-Lₚ and Lᵢ-I exist in a dynamic equilibrium in vivo where the conjugate P-L-I exists in molar excess of at least one of the conjugate parts P-Lₚ and Lᵢ-I, and further characterised in that
   b. at least one of the conjugate parts P-Lₚ and Lᵢ-I binds covalently to glucose, whereby the concentration of P that is not bound to I increases in vivo when the concentration of glucose increases in vivo, or, alternatively further characterised in that the hydrolysis of the hydrolysable linker L is being promoted by glucose.

In a highly preferred embodiment, the present invention relates to the use thereof in the treatment of a human subject. In a highly preferred embodiment, the present invention relates to the use thereof in the treatment of diabetes in a human subject. In a highly preferred embodiment, the present invention relates to the use thereof in the manufacture of a medicament for the treatment of diabetes in a human subject.

### EXAMPLES

Example 1 exemplifies the synthesis of exemplary hydrolysable linker (L) molecules.

Example 2 exemplifies a procedure for forming a linker (L) with handles ready for grafting of a peptide (P) and an inhibitor (I).

Example 3 exemplifies the synthesis of the linker attached to an inhibitor (I). In this example, the inhibitor or inactivator complex (I) is a C18 fatty acid, which does not in itself inhibit the activity of the peptide (see example 4) but is known to bind to albumin in vivo. Thus, in vivo inactivation is ultimately achieved by the inhibitor binding and clustering conjugates to albumin.

Example 4 exemplifies the synthesis of a reference peptide hormone conjugated to an inactivator (I), without a hydrolysable linker. The example shown is Lys^{B29}N^{ϵ}-octadecanoyl human insulin.

Example 5 exemplifies the synthesis of an insulin conjugate according to the invention.

Example 6 analyses the exemplary hydrolysable linker (L) molecules 1-19 of example 1 for their ability to hydrolyse in vitro and subsequently bind glucose.

Example 7 evaluates the reaction rate of three different linkers (linkers 1, 14 and 15) at various glucose concentrations, i.e. their ability to hydrolyse and react with glucose to form a linker glucose compound.

Example 8 evaluates the hydrolysability of the linker attached to insulin (conjugate 2 of example 5), in the presence of glucose.

Example 9_evaluates the in vitro potency on the insulin B receptor of human insulin, insulin conjugates 1 and 2 of example 5 (conjugate 1 without inhibitor (I), conjugate 2 with inhibitor (I)), and reference insulin conjugated to inhibitor (I) without linker from example 4.

Example 10 evaluates human insulin conjugated with a C18 fatty acid of example 4 and its ability to interact with albumin and reduce insulin activity, measured by scITT in lean rats.

### Example 1: Synthesis of Hydrolysable Linker Molecules—Hydrazones

General Procedure:

A hydrazide (1 equiv) was dissolved in methanol into which an aldehyde (1 equiv) and catalytic amounts of acetic acid was added. The mixture was heated to reflux. The reaction was followed by TLC (thin-layer chromatography). The solvent was removed in vacuo yielding the crude product as an oil or as solid. The individual purifications conditions are for each molecule listed below.

### Linker 1. ((E)-N′-(3-(benzyloxy)propylidene)-4-methoxybenzohydrazide)

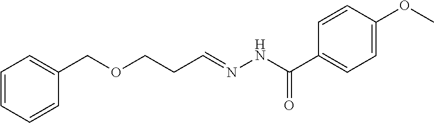 [SMILES: COc1ccc(C(=O)N/N=C/CCOCc2ccccc2)cc1]

Hydrazide: 4-methoxybenzohydrazide (CAS number: 3290-99-1)

Aldehyde: 3-benzyloxypropionaldehyde (CAS number: 19790-60-4)

Purification Method:

Purified by column chromatography 0-3% methanol/dichloromethane.

¹H NMR (300 MHz, DMSO-d₆) δ 11.35 (s, 1H, NH), 7.84 (d, J=8.8 Hz, 2H, C2′H, C6′H), 7.78 (m, 1H, CHN), 7.37-7.25 (m, 5H, Ph), 7.02 (d, J=8.8 Hz, 2H, C3′H, C5′H), 4.50 (s, 2H, PhCH₂O), 3.82 (s, 3H, OCH₃), 3.65 (t, J=6.3 Hz, 2H, OCH₂), 2.55 (q, J=6.0 Hz, 2H, CH₂CHN).

¹³C NMR (75 MHz, DMSO-d₆) δ 162.2 (C4′OCH₃), 161.82 (CO), 149.27 (CHN), 138.34 (C1), 129.37 (C2′, C6′), 128.22 (C3, C5), 127.9 (C2, C6), 127.39 (C4), 125.49 (C1′), 113.57 (C3′, C5′), 71.85 (Ar—CH₂O), 66.97 (OCH₂), 55.34 (OCH₃), 32.67 (CH₂CHN). HRMS (ESI): m/z: calcd. for C₁₈H₂₁N₂O₃: 313.1552 [M+H]⁺; found 313.1548.

### Linker 2. ((E)-N′-(3-(benzyloxy)propylidene)benzohydrazide)

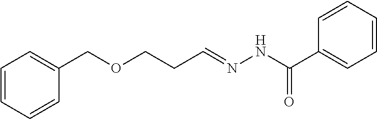 [SMILES: O=C(N/N=C/CCOCc1ccccc1)c1ccccc1]

Hydrazide: Benzohydrazide (CAS number: 613-94-5)

Aldehyde: 3-benzyloxypropionaldehyde (CAS number: 19790-60-4)

Purification Method:

Purified by column chromatography 0.5-1% methanol/dichloromethane.

¹H NMR (300 MHz, DMSO-d₆) δ 11.49 (s, 1H, NH), 7.85 (d, J=7.1 Hz, 2H, C2′H, C6′H) 7.80 (t, J=5.9 Hz, 1H, CHN), 7.56 (d, J=7.1 Hz, 2H, C3′H, C5′H), 7.5 (m, 1H, C4′H), 7.37-7.25 (m, 5H, C2-6H), 4.50 (s, 2H, Ar—CH₂O), 3.66 (t, J=6.1 Hz, 2H, OCH₂), 2.55 (m, 2H, CH₂CHN).

¹³C NMR (75 MHz, DMSO-d₆) δ 162.75 (CO), 150.0 (CHN), 138.33 (C1), 133.51 (C4′), 128.35 (C3, C5), 128.22 (C3′, C5′), 127.47 (C2, C6, C2′, C6′), 127.40 (C4), 126.36 (C1′), 71.84 (Ar—CH₂O), 66.91 (OCH₂), 32.69 (CH₂CHN). HRMS (ESI): m/z: calcd. for C₁₇H₁₉N₂O₂: 283.1447 [M+H]⁺; found 283.1439.

### Linker 3. ((E)-N′-(3-(benzyloxy)propylidene)-1-hydroxy-2-naphthohydrazide)

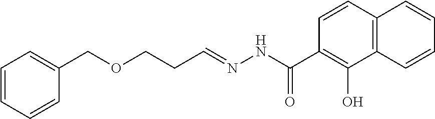 [SMILES: O=C(N/N=C/CCOCc1ccccc1)c1ccc2ccccc2c1O]

Hydrazide: 1-hydroxy-2-naphthohydrazide (CAS number: 7732-44-7)

Aldehyde: 3-benzyloxypropionaldehyde (CAS number: 19790-60-4)

Purification Method:

Purified by column chromatography 0-5% methanol/dichloromethane.

¹H NMR (300 MHz, DMSO-d₆) δ 14.20 (C1′-OH), 11.79 (s, 1H, NH), 8.30 (m, 1H, C5′H) 7.98-7.87 (m, 3H, C8′H, C4′H, CHN), 7.71-7.54 (m, 2H, C6′H, C7′H), 7.48-7.18 (m, 6H, C3′H, C2-6H), 4.53 (s, 2H, Ar—CH₂O), 3.70 (t, J=6.3 Hz, 2H, OCH₂), 2.67-2.58 (m, 2H, CH₂CHN).

¹³C NMR (75 MHz, DMSO-d₆) δ 166.86 (CO), 160.20 (C1′-OH), 153.1 (CHN), 138.32 (C1), 135.85 (C9′), 129.09 (C7′), 128.22 (C3, C5), 127.96 (C8′), 127.49 (C2, C6), 127.41 (C4), 126.56 (C10′), 126.36 (C6′), 125.9 (C2′), 124.61 (C5′), 122.94 (C4′), 117.68 (C3′), 71.90 (Ar—CH₂O), 66.79 (OCH₂), 32.79 (CH₂CHN). ESI: m/z: calcd. for C₂₁H₂₁N₂O₃: 349.1552 [M+H]⁺; found 349.0.

### Linker 4. ((E)-N′-(2,4,6-trihydroxybenzylidene)benzohydrazide)

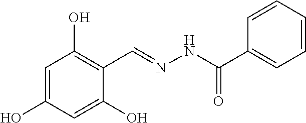 [SMILES: O=C(N/N=C/c1c(O)cc(O)cc1O)c1ccccc1]

Hydrazide: Benzohydrazide (CAS number: 613-94-5)

Aldehyde: 2,4,6-trihydroxybenzaldehyde (CAS number: 487-70-7)

Purification Method:

After removal of solvent the generated solid was washed with cold methanol giving the product as a brown to orange powder.

¹H NMR (300 MHz, DMSO-d₆) δ 11.93 (s, 1H, NH), 11.14 (s, 2H, C2-OH, C6-OH), 9.85 (s, 1H, C4-OH), 8.87 (s, 1H, CHN), 8.05-7.95 (m, 2H, C2′H, C6′H), 7.70-7.50 (m, 3H, C3′H, C4′H, C5′H), 5.91 (s, 2H, C3H, C5H).

¹³C NMR (75 MHz, DMSO-d₆) δ 162.09 (C4), 161.52 (CO), 159.67 (C2, C6), 146.80 (CHN), 132.87 (C4′), 131.70 (C1′), 128.45 (C3′, C5′), 127.45 (C2′, C6′), 99.03 (C1), 94.34 (C3, C5). HRMS (ESI): m/z: calcd. for C₁₄H₁₃N₂O₄: 272.0875 [M+H]⁺; found 273.0867.

### Linker 5. ((E)-N′-(2-hydroxybenzylidene)-4-nitrobenzohydrazide)

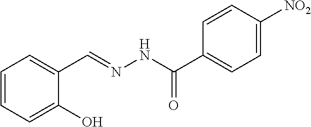 [SMILES: O=C(N/N=C/c1ccccc1O)c1ccc([N+](=O)[O-])cc1]

Hydrazide: 4-nitrobenzohydrazide (CAS number: 636-97-5)

Aldehyde: Salicylic aldehyde (CAS number: 90-02-8)

Purification Method:

A white precipitate was formed during reflux, and the crude precipitate was filtrated and washed with cold methanol giving the product a pale, yellow powder.

¹H NMR (300 MHz, DMSO-d₆) δ 12.35 (s, 1H, NH), 11.10 (s, 1H, C2-OH), 8.69 (s, 1H, CHN), 8.40 (dt, 2H, J=8.9, 2.0 Hz, 2H, C3H, C5H), 8.18 (dt, 2H, J=8.9, 2.7 Hz, C2H, C6H), 7.60 (dd, J=7.7, 1.4 Hz, 1H, C6H), 7.32 (ddd, J=7.5, 1.8 Hz, 1H, C4H), 6.95 (d, J=7.7 Hz, 1H, C3H), 6.94-6.90 (m, 1H, C5H).

¹³C NMR (75 MHz, DMSO-d₆) δ 161.18 (CO), 157.46 (C2-OH), 149.36 (C4′-NO₂), 148.93 (CHN), 138.53 (C1′), 131.68 (C4), 129.23 (C2′, C6′), 129.16 (C6), 123.67 (C3′, C5′), 119.39 (C5), 118.66 (C1), 116.41 (C3). HRMS (ESI): m/z: calcd. for C₁₄H₁₁N₃O₄Na: 308.0647 [M+Na]⁺; found 308.0639.

### Linker 6. ((E)-4-methoxy-N′-(2-nitrobenzylidene)benzohydrazide)

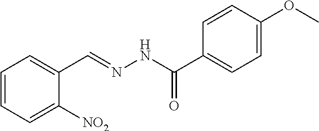 [SMILES: COc1ccc(C(=O)N/N=C/c2ccccc2[N+](=O)[O-])cc1]

Hydrazide: 4-methoxybenzohydrazide (CAS number: 3290-99-1)

Aldehyde: 2-nitrobenzaldehyde (CAS number: 552-89-6)

Purification Method:

Pale yellow needles precipitated after cooling the reaction mixture to room temperature. The precipitate was filtrated and washed with cold methanol to give the desired product.

¹H NMR (300 MHz, DMSO-d₆) δ 12.07 (s, 1H, NH), 8.86 (s, 1H, CHN), 8.13 (d, J=7.4 Hz, C6H), 8.08 (dd, J=8.2, 1.1 Hz, 1H, C3H), 7.94 (d, J=8.9 Hz, 2H, C2′H, C6′H), 7.82 (t, J=7.4 Hz, 1H, C5H), 7.72-7.62 (m, 1H, C4H), 7.08 (d, J=8.9 Hz, 2H, C3′H, C5′H), 3.85 (s, 3H, CH₃)

¹³C NMR (75 MHz, DMSO-d₆) δ 162.56 (CO), 161.71 (C4′-OCH₃) 148.19 (C2-NO₂), 142.04 (CHN), 133.64 (C5), 130.49 (C4), 129.71 (C2′, C6′), 128.79 (C1), 127.83 (C6), 124.98 (C1′), 124.59 (C3), 113.71 (C3′, C5′), 55.43 (CH₃). HRMS (ESI): m/z: calcd. for C₁₅H₁₄N₃O₄: 300.0984 [M+H]⁺; found 300.0975.

### Linker 7. ((E)-2,4-dihydroxy-N′-(2-nitrobenzylidene)benzohydrazide)

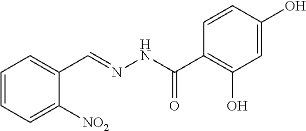 [SMILES: O=C(N/N=C/c1ccccc1[N+](=O)[O-])c1ccc(O)cc1O]

Hydrazide: 2,4-dihydroxybenzohydrazide (CAS number: 13221-86-8)

Aldehyde: 2-nitrobenzaldehyde (CAS number: 552-89-6)

Purification Method:

A yellow precipitate was formed during reflux. The reaction mixture was filtrated and washed with cold methanol giving the product a pale, yellow powder.

¹H NMR (300 MHz, DMSO-d₆) δ 12.25 (s, 1H, NH), 12.01 (s, 1H, C6′-OH), 10.27 (s, 1H, C4′-OH), 8.84 (s, 1H, CHN), 8.13 (d, J=7.5 Hz, C5H), 8.09 (dd, J=8.3, 1.1 Hz, 1H, C3H), 7.87-7.78 (m, 2H, C4H, C2′H), 7.69 (ddd, J=7.2, 1.5 Hz, 1H, C4H), 6.38 (dd, J=8.7, 2.4 Hz, 1H), 6.33 (d, J=2.4 Hz, 1H)

¹³C NMR (75 MHz, DMSO-d₆) δ 165.89 (CO), 162.94 (C4′-OH), 162.47 (C6′-OH), 148.23 (C2-NO₂), 142.89 (CHN), 133.69 (C5), 130.68 (C2′), 128.59 (C6), 127.96 (C1), 124.63 (C3), 108.68 (C1′), 107.48 (C3′), 102.84 (C5′). HRMS (ESI): m/z: calcd. for C₁₄H₁₂N₃O₅: 302.0777 [M+H]⁺; found 302.0767.

### Linker 8. ((E)-N′-(3-(benzyloxy)-2-methylpropylidene)-4-methoxybenzohydrazide)

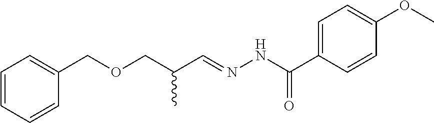 [SMILES: COc1ccc(C(=O)N/N=C/C(C)COCc2ccccc2)cc1]

Hydrazide: 4-methoxybenzohydrazide (CAS number: 3290-99-1)

Aldehyde: (R,S)-3-benzyloxy-2-methylpropionaldehyde (CAS number: 73814-73-0)

Purification Method:

After the solvent was removed in vacuo, a clear oil was formed. The product slowly (overnight) precipitated from the oil as white needles, which were washed with heptane to yield the desired product.

¹H NMR (300 MHz, DMSO-d₆) δ 11.33 (s, 1H, NH), 7.85 (d, 2H, J=8.9 Hz, C2′H, C6′H), 7.75 (d, 1H, J=4.9 Hz, CHN), 7.37-7.28 (m, 5H, Ph), 7.02 (d, 2H, J=8.8 Hz, C3′H, C5′H), 4.5 (s, 2H, CH2O), 3.52, 3.49 (dd, 2H, J=9.1 Hz, OCH2CH), 2.77-2.68 (m, 1H, CH₂CHCH₃), 1.09 (d, J=6.9 Hz, CH₃).

¹³C NMR (75 MHz, DMSO-d₆) δ 161.78 (CO, C4′), 153.25 (CHN), 138.37 (C1), 129.35 (C2′, C6′), 128.22 (C3, C5), 127.41 (C2, C6), 127.38 (C4), 125.56 (C1′), 113.57 (C3′, C5′), 72.60 (OCH₂CH), 72.05 (PhCH₂O), 55.34 (OCH₃), 36.82 (CH₂CHCH₃), 14.51 (CH₃). HRMS (ESI): m/z: calcd. for C₁₉H₂₃N₂O₃: 327.1709 [M+H]⁺; found 327.1705.

### Linker 9. ((E)-N′-(3-(benzyloxy)-2-methylpropylidene)-2,4-dihydroxybenzohydrazide)

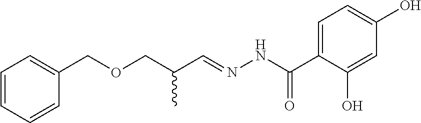 [SMILES: CC(/C=N/NC(=O)c1ccc(O)cc1O)COCc1ccccc1]

Hydrazide: 2,4-dihydroxybenzohydrazide (CAS number: 13221-86-8)

Aldehyde: (R,S)-3-benzyloxy-2-methylpropionaldehyde (CAS number: 73814-73-0)

Purification Method:

Column chromatography 40-60% ethyl acetate/heptane.

¹H NMR (300 MHz, DMSO-d₆) δ 12.45 (s, 1H, NH), 11.3 (s, 1H, C2-OH), 10.2 (s, 1H, C4-OH), 7.76-7.70 (m, 2H, CHN, C6H), 7.38-7.25 (m, 5H, Ph′), 6.32 (dd, 1H, J=8.7 Hz, J=2.4 Hz, C5H) 6.27 (d, 1H, J=2.4 Hz, C3H), 4.51 (s, 2H Ar′CH₂), 3.5 (dd, 2H, J=9.3 Hz, OCH₂CH), 2.78-2.68 (m, 1H, CH₂CHCH₃), 1.09 (d, 3H, J=6.9 Hz, CH₃).

¹³C NMR (75 MHz, DMSO-d₆) δ 162.52 (CO), 162.5 (C4′-OH, C2′-OH), 154.38 (CHN), 138.34 (C1), 129.31 (C6′), 128.22 (C2, C6), 127.43 (C3, C5), 129.39 (C4), 107.16 (C5′), 105.81 (C1′), 102.79 (C3′), 72.49 (OCH₂), 72.06 (PhCH₂O), 36.85 (CH₂CHCH₃), 14.40 (CH₃). HRMS (ESI): m/z: calcd. for C₁₈H₂₀N₂O₄: 329.1501 [M+H]⁺; found 329.14988.

### Linker 10. ((E)-N′-(3-(benzyloxy)propylidene)-2,4-dihydroxybenzohydrazide)

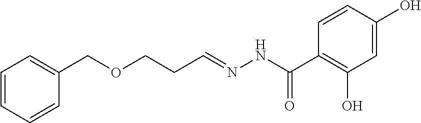 [SMILES: O=C(N/N=C/CCOCc1ccccc1)c1ccc(O)cc1O]

Hydrazide: 2,4-dihydroxybenzohydrazide (CAS number: 13221-86-8)

Aldehyde: 3-benzyloxypropionaldehyde (CAS number: 19790-60-4)

Purification Method:

Column chromatography 1-3% methanol/dichloromethane.

¹H NMR (300 MHz, DMSO-d₆) δ 12.42 (s, 1H, C2-OH), 11.4 (s, 1H, NH), 10.17 (s, 1H, C4-OH), 7.78-7.70 (m, 2H, CHN, C6H), 7.40-7.20 (m, 5H, Ph′), 6.32 (dd, 1H, J=9 Hz, J=3 Hz, C5H), 6.28 (d, 1H, J=3 Hz, C3H), 4.50 (s, 2H Ar′CH₂O), 3.66 (t, 2H, J=6 Hz, OCH₂CH), 2.51 (m, 1H, CH₂CHCHN).

¹³C NMR (75 MHz, DMSO-d₆) δ 165.3 (CO), 162.5 (C4′-OH, C2′-OH), 150.6 (CHN), 138.3 (C1), 128.17 (C4), 128.22 (C2, C6), 128.0 (C6′), 127.5 (C3, C5), 107.16 (C5′), 105.8 (C1′), 102.80 (C3′), 71.9 (PhCH₂O), 66.8 (OCH₂), 32.9 (CH₂CHCHN).

### Linker 11. ((E)-N′-(3-(benzyloxy)propylidene)-4-(dimethylamino)benzohydrazide)

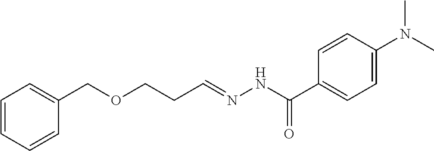 [SMILES: CN(C)c1ccc(C(=O)N/N=C/CCOCc2ccccc2)cc1]

Hydrazide: 4-(dimethylamino)benzohydrazide (CAS number: 19353-92-5)

Aldehyde: 3-benzyloxypropionaldehyde (CAS number: 19790-60-4)

Purification Method:

Column chromatography 1-3% methanol/dichloromethane.

¹H NMR (300 MHz, DMSO-d₆) δ 11.17 (s, 1H, NH), 7.76 (d, 2H, J=9 Hz, C2H, C6H), 7.75 (t, J=5.5 Hz, 1H, CHN), 7.36-7.27 (m, 5H, Ph′), 6.72 (d, 2H, J=9 Hz, C3H, C5H), 4.51 (s, 2H Ar′CH₂O), 3.65 (t, 2H, J=6 Hz, OCH₂CH), 2.98 (s, 6H, N(CH₃)₂), 2.55 (q, 2H, J=5.5 Hz, CH₂CHCHN).

¹³C NMR (75 MHz, DMSO-d₆) δ 162.6 (CO), 152.3 (C4′N(CH₃)₂), 148.0 (CHN), 138.4 (C1), 128.9 (C4), 128.2 (C2, C6, C2′, C6′), 127.5 (C3, C5), 119.6 (C1′), 110.7 (C3′, C5′), 71.9 (PhCH₂O), 67.1 (OCH₂), 42.1 (N(CH₃)₂), 32.7 (CH₂CHCH₃). HRMS (ESI): m/z: calcd. for C₁₉H₂₄N₃O₂: 326.18685 [M+H]⁺; found 326.1867.

### Linker 12. ((E)-N′-(2-(benzyloxy)ethylidene)-2,4-dihydroxybenzohydrazide)

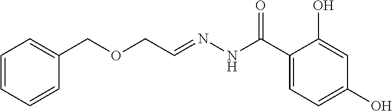 [SMILES: O=C(N/N=C/COCc1ccccc1)c1ccc(O)cc1O]

Hydrazide: 2,4-dihydroxybenzohydrazide (CAS number: 13221-86-8)

Aldehyde: Benzyloxyacetaldehyde (CAS number: 60656-87-3)

Purification Method:

Column chromatography 30-80% ethyl acetate/heptane

¹H NMR (300 MHz, DMSO-d₆) δ 12.25 (s, 1H, C2-OH), 11.5 (s, 1H, NH), 10.2 (s, 1H, C4-OH), 7.81 (t, 1H, J=5 Hz, CHN), 7.74 (d, 1H, J=8.7 Hz, C6H), 7.39-7.27 (m, 5H, Ph), 6.35 (dd, 1H, J=8.7 Hz, J=2.4 Hz, C5H) 6.30 (d, 1H, J=2.4 Hz, C3H), 4.55 (s, 2H Ar′CH₂O), 4.18 (d, 2H, J=5 Hz, OCH₂CHN).

¹³C NMR (75 MHz, DMSO-d₆) δ 165.8 (CO), 162.7 (C2′-OH), 162.3 (C4′-OH), 148.4 (CHN), 137.9 (C1), 129.6 (C4), 128.3 (C6′), 128.1 (C3, C5), 127.7 (C2, C6), 107.3 (C5′), 105.9 (C1′), 102.8 (C3′), 71.8 (PhCH₂O), 69.1 (OCH₂CHN). HRMS (ESI): m/z: calcd. for C₁₆H₁₆N₂O₄Na: 323.10078 [M+Na]⁺; found 323.10075.

### Linker 13. ((E)-N′-(2-(benzyloxy)ethylidene)-4-(dimethylamino)benzohydrazide)

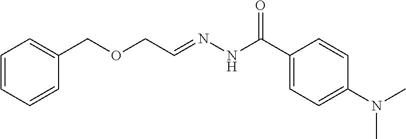 [SMILES: CN(C)c1ccc(C(=O)N/N=C/COCc2ccccc2)cc1]

Hydrazide: 4-(dimethylamino)benzohydrazide (CAS number: 19353-92-5)

Aldehyde: Benzyloxyacetaldehyde (CAS number: 60656-87-3)

Purification Method:

The desired product precipitated as a white powder during cooling of the reaction mixture.

¹H NMR (300 MHz, DMSO-d₆) δ 11.3 (s, 1H, NH), 7.76 (d, 1H, J=9 Hz, C2H, C6H), 7.75 (m, 1H, CHN), 7.38-7.27 (m, 5H, Ph), 6.72 (d, 1H, J=9 Hz, C3H, C5H), 4.54 (s, 2H Ar′CH₂O), 4.16 (d, 2H, J=5 Hz, OCH₂CHN), 2.98 (s, 6H, N(CH₃)₂).

¹³C NMR (75 MHz, DMSO-d₆) δ 164.7 (CO), 152.4 (C4′-N(CH₃)₂), 148.4 (CHN), 138.0 (C1), 129.2 (C4), 128.3 (C6), 128.1 (C3, C5), 127.6 (C2, C6), 127.5 (C2′, C6′), 119.3 (C1′), 110.8 (C3′, C5′), 71.7 (PhCH₂O), 69.2 (OCH₂CHN), 39.6 (N(CH₃)₂). HRMS (ESI): m/z: calcd. for C₁₈H₂₂N₃O₂: 312.1712 [M+H]⁺; found 312.1798.

### Linker 14. ((E)-N′-(2-(benzyloxy)ethylidene)-4-methoxybenzohydrazide)

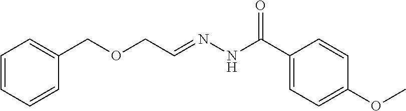 [SMILES: COc1ccc(C(=O)N/N=C/COCc2ccccc2)cc1]

Hydrazide: 4-methoxybenzohydrazide (CAS number: 3290-99-1)

Aldehyde: Benzyloxyacetaldehyde (CAS number: 60656-87-3)

Purification Method:

Purified by column chromatography 0-2% methanol/dichloromethane.

¹H NMR (300 MHz, DMSO-d₆) δ 11.52 (s, 1H, NH), 7.86 (d, J=8.7 Hz, 2H, C2′H, C6′H), 7.81 (m, 1H, CHN), 7.39-7.25 (m, 5H, Ph), 7.04 (d, J=8.7 Hz, 2H, C3′H, C5′H), 4.54 (s, 2H, PhCH₂O), 4.17 (d, 2H, J=5.1 Hz, OCH₃CHN), 3.84 (s, 3H, OCH₃).

¹³C NMR (75 MHz, DMSO-d₆) δ 168.1 (C4′OCH₃), 161.9 (CO), 147.6 (CHN), 138.0 (C1), 129.5 (C4), 128.3 (C2′, C6′), 128.2 (C3, C5), 127.6 (C2, C6), 120.7 (C1′), 115.6, 113.6 (C3′, C5′), 71.7 (PhCH₂O), 66.1 (OCH₂CHN), 55.4 (OCH₃). HRMS (ESI): m/z: calcd. for C₁₇H₁₉N₂O₃: 299.1396 [M+H]⁺; found 299.1404.

### Linker 15. ((E)-N′-(2-(benzyloxy)ethylidene)-2-hydroxy-4-methoxybenzohydrazide)

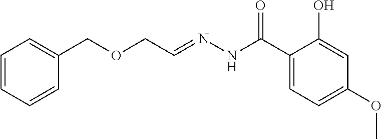 [SMILES: COc1ccc(C(=O)N/N=C/COCc2ccccc2)c(O)c1]

Hydrazide: 2-hydroxy-4-methoxybenzohydrazide (CAS number: 41697-08-9)

Aldehyde: Benzyloxyacetaldehyde (CAS number: 60656-87-3)

Purification Method:

Purified by column chromatography 0-0.2% methanol/dichloromethane.

¹H NMR (300 MHz, DMSO-d₆) δ 12.39 (s, 1H, C2-OH), 11.6 (s, 1H, NH), 7.83 (d, 1H, J=9 Hz, C6′H), 7.83 (m, 1H, CHN), 7.38-7.27 (m, 5H, Ph), 6.53 (dd, 1H, J=9 Hz, J=2.4 Hz, C5′H) 6.48 (d, 1H, J=2.4 Hz, C3′H), 4.55 (s, 2H Ph′CH₂O), 4.19 (d, 2H, J=5.1, OCH₂CHN), 3.77 (s, 3H, OCH₃).

¹³C NMR (75 MHz, DMSO-d₆) δ 165.5 (CO), 163.9 (C4′-OCH₃), 162.3 (C2′-OH), 148.7 (CHN), 137.9 (C1), 129.4 (C6′), 128.3 (C6′), 127.7 (C2, C6), 127.6 (C3, C5), 107.1 (C1′), 106.3 (C3′), 101.3 (C5′), 71.8 (PhCH₂O), 69.0 (OCH₂CHN), 55.4 (OCH₃). HRMS (ESI): m/z: calcd. for C₁₇H₁₈N₂O₄: 315.1345 [M+H]⁺; found 315.1346.

### Linker 16. ((E)-4-methoxy-N′-(benzylidene)benzohydrazide)

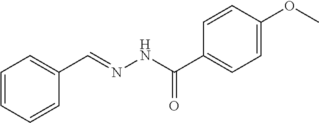 [SMILES: COc1ccc(C(=O)N/N=C/c2ccccc2)cc1]

Hydrazide: 4-methoxybenzohydrazide (CAS number: 3290-99-1)

Aldehyde: Benzaldehyde (CAS number: 100-52-7) Synthesised according to Taha et al., Molecules, 2014, 19 (1), 1286-1301.

### Linker 17. ((E)-2,4-dihydroxy-N′-(benzylidene)benzohydrazide)

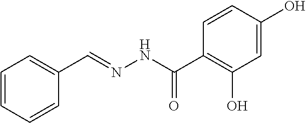 [SMILES: O=C(N/N=C/c1ccccc1)c1ccc(O)cc1O]

Hydrazide: 2,4-dihydroxybenzohydrazide (CAS number: 13221-86-8)

Aldehyde: Benzaldehyde (CAS number: 100-52-7) Synthesised according to B. Camber and D. D. Dziewiatkowski, JACS, 1951, 73 (8), 4021-4021.

### Linker 18. ((E)-4-amino-N′-(benzylidene)benzohydrazide)

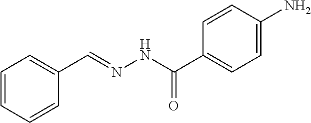 [SMILES: Nc1ccc(C(=O)N/N=C/c2ccccc2)cc1]

Hydrazide: 4-amino-benzohydrazide (CAS number: 5351-17-7)

Aldehyde: Benzaldehyde (CAS number: 100-52-7) Synthesised according to Adeniyi et al., Pakistan J. Sci. Industrial Res., 2006, 49 (4), 246-250.

### Linker 19. ((E)-4-dimethylamino-N′-(benzylidene)benzohydrazide)

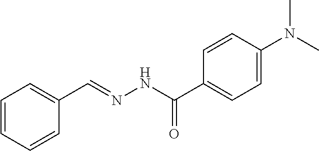 [SMILES: CN(C)c1ccc(C(=O)N/N=C/c2ccccc2)cc1]

Hydrazide: 4-(dimethylamino)benzohydrazide (CAS number: 19353-92-5)

Aldehyde: Benzaldehyde (CAS number: 100-52-7) Synthesised according to Wen et al., Chem. Commun., 2006, 106-108.

### Example 2: Synthesis Procedure for Linker (L) 20 with Handles Prepared for Grafting of Peptide (P) and Inhibitor (I)

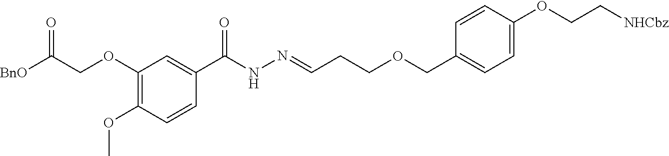 [SMILES: COc1ccc(C(=O)N/N=C/CCOCc2ccc(OCCNC(=O)OCc3ccccc3)cc2)cc1OCC(=O)OCc1ccccc1]

Synthesis of Intermediate Compound 22

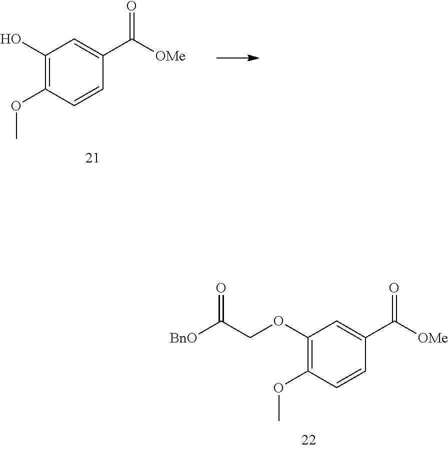 [SMILES: COC(=O)c1ccc(OC)c(O)c1 ; COC(=O)c1ccc(OC)c(OCC(=O)OCc2ccccc2)c1]

Methyl 3-hydroxy-4-methoxybenzoate (21) (0.956 g, 5.19 mmol) was dissolved in dimethylformamide (10 mL). K₂CO₃ (potassium carbonate) (1.44 g, 10.4 mmol), and methyl bromoacetate (1.45 mL, 5.71 mmol) was added, and the reaction mixture was stirred at room temperature for 24 h. The residue was filtered and concentrated, re-dissolved in ethyl acetate and washed with 1M NaOH (sodium hydroxide), brine and dried with MgSO₄ (magnesium sulfate). Purification by silica gel chromatography (hexane:ethyl acetate 3:1) gave compound 22 (1.61 g, 4.88 mmol, 94%). MS (ESI): m/z calcd for C₁₈H₁₈O₆[M+H]⁺ 331.11; found 331.57.

Synthesis of Intermediate Compound 23

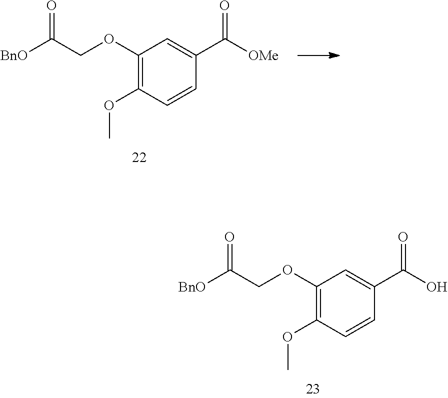 [SMILES: COC(=O)c1ccc(OC)c(OCC(=O)OCc2ccccc2)c1 ; COc1ccc(C(=O)O)cc1OCC(=O)OCc1ccccc1]

Compound 22 (0.983 g, 2.98 mmol) was dissolved in tetrahydrofuran/methanol/water 1:1:1 (9 mL). 2M NaOH (1.5 mL) was added and the reaction was stirred at room temperature for 30 min. The reaction was made acidic by addition of 1M HCl (hydrogen chloride), concentrated and re-dissolved in ethyl acetate. The organic phase was washed with water, brine, dried with MgSO₄ filtered and evaporated. The product, compound 23 (0.914 g, 2.89 mmol, 97%), was used in the next reaction without further purification. MS (ESI): m/z calcd for C₁₁H₁₂O₆[M+Na]⁺ 339.06; found 339.36.

Synthesis of Intermediate Compound 24

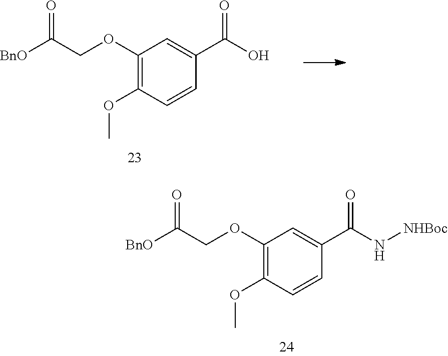 [SMILES: COc1ccc(C(=O)O)cc1OCC(=O)OCc1ccccc1 ; COc1ccc(C(=O)NNC(=O)OC(C)(C)C)cc1OCC(=O)OCc1ccccc1]

Compound 23 (60 mg, 0.189 mmol) was dissolved in dichloromethane and cooled to 0° C. Oxalyl chloride (51 μL, 0.378 mmol) was added, and the reaction was stirred at 0° C. for 1 h and at room temperature for 1 h. The solvent was evaporated, and the residue was re-dissolved in dichloromethane. Tert-butyl carbazate (NH₂NHBoc) (50 mg, 0.378 mmol) and Et₃N (triethylamine) (53 μL, 0.378 mmol) were added, and the reaction was stirred at room temperature for 4 h. Evaporation and purification by silica gel chromatography (hexane/ethyl acetate 1:1) gave compound 24 (50 mg, 0.116 mmol, 61%).

Synthesis of Intermediate Compound 25

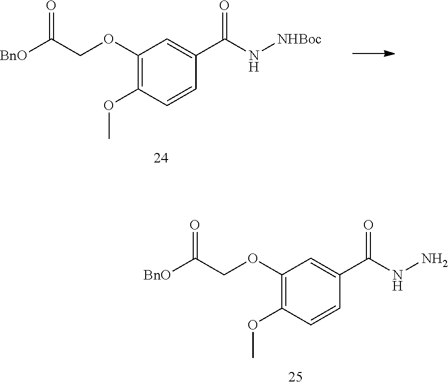 [SMILES: COc1ccc(C(=O)NNC(=O)OC(C)(C)C)cc1OCC(=O)OCc1ccccc1 ; COc1ccc(C(=O)NN)cc1OCC(=O)OCc1ccccc1]

Compound 24 (25 mg, 0.058 mmol) was dissolved in dichloromethane (5 mL). TFA (trifluoroacetic acid) (200 μL) was added, and the reaction was stirred at room temperature for 2 h. Evaporation gave compound 25, and it was used in the next reaction without further purification.

Synthesis of Intermediate Compound 27

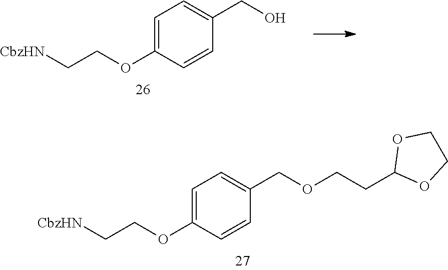 [SMILES: O=C(NCCOc1ccc(CO)cc1)OCc1ccccc1 ; O=C(NCCOc1ccc(COCCC2OCCO2)cc1)OCc1ccccc1]

Benzyl 2-[4-(hydroxymethyl)phenoxy]ethylcarbamate (Compound 26, synthesised according to procedure described in ChemBioChem, 2005, 6, 2271-2280) was dissolved in dimethylformamide and added dropwise to NaH (sodium hydride) in dimethylformamide equipped with a N₂-atmosphere at 0° C. The reaction mixture was stirred at 0° C., where after 2-(2-bromoethyl)-1,3-dioxolane was added dropwise and stirred for another 4 h at room temperature. The reaction was quenched by addition of water and extracted with ethyl acetate. The organic phase was dried over Na₂SO₄ (sodium sulfate), filtrated and concentrated in vacuo. Purification by silica gel chromatography (0-100% ethyl acetate in hexane) to give compound 27.

Synthesis of Linker Compound 20

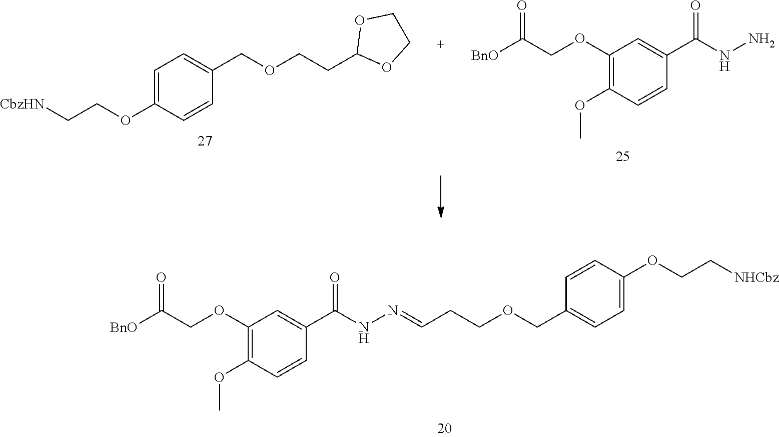 [SMILES: O=C(NCCOc1ccc(COCCC2OCCO2)cc1)OCc1ccccc1 ; COc1ccc(C(=O)NN)cc1OCC(=O)OCc1ccccc1 ; COc1ccc(C(=O)N/N=C/CCOCc2ccc(OCCNC(=O)OCc3ccccc3)cc2)cc1OCC(=O)OCc1ccccc1]

Compound 25 and compound 27 were dissolved in methanol. Acetic acid was added, and the reaction was stirred at room temperature for 24 h. The product 20 was detected by MS (ESI): m/z calcd. for C₃₇H₃₉N₃O₉ [M+H]⁺ 670.27; found 671.32.

### Example 3: Synthesis of Linker and Inactivator Complex

Synthesis of Intermediate Compound 29

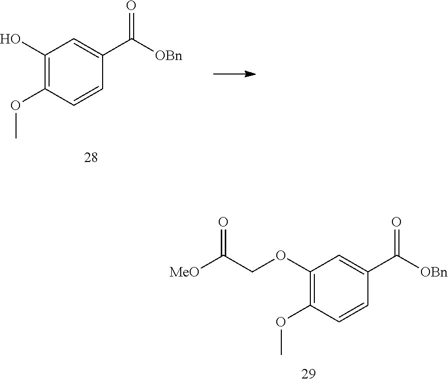 [SMILES: COc1ccc(C(=O)OCc2ccccc2)cc1O ; COC(=O)COc1cc(C(=O)OCc2ccccc2)ccc1OC]

Benzyl 3-hydroxy-4-methoxybenzoate (28) (0.70 g, 2.71 mmol) was dissolved in dimethylformamide (10 mL). K₂CO₃ (0.75 g, 5.42 mmol) and methyl bromoacetate (0.26 mL, 2.71 mmol) were added, and the reaction mixture was stirred at room temperature for 24 h. The residue was filtered and concentrated, re-dissolved in ethyl acetate and washed with 1M NaOH, brine, dried with MgSO₄, filtered and evaporated. Purification by silica gel chromatography (hexane/ethyl acetate 3:1) gave compound 29 (0.72 g, 2.18 mmol, 80%).

¹H NMR (300 MHz, CDCl₃) δ 7.77 (dd, 1H, ArH), 7.52 (d, 1H, ARH), 7.33-7.44 (m, 5H, ArH), 6.92 (d, 1H, ArH), 5.33 (s, 2H, OCH₂Ar), 4.73 (s, 2H, OCH₂C═O), 3.94 (s, 3H, OCH₃), 3.79 (s, 3H, O═C—OCH₃); MS (ESI): m/z calcd for C₁₈H₁₈O₆[M+H]⁺ 331.11; found 331.46.

### Synthesis of Intermediate Compound 30

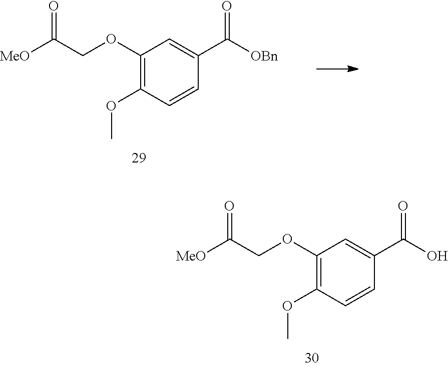 [SMILES: COC(=O)COc1cc(C(=O)OCc2ccccc2)ccc1OC ; COC(=O)COc1cc(C(=O)O)ccc1OC]

Compound 29 (121 mg, 0.366 mmol) was dissolved in methanol (5 mL) and flushed with N₂-gas. 10% Pd/C (10% wt, 4 mg, 0.037 mmol) was added followed by the portion wise addition of NaBH₄ (sodium borohydride) (21 mg, 0.549 mmol). The reaction mixture was stirred at room temperature for 30 min and filtered through Celite. The filtrate was made acidic by addition of 1M HCl, concentrated and re-dissolved in ethyl acetate. The organic phase was washed with water, brine, dried with MgSO₄, filtered and evaporated. The product, compound 30 (85 mg, 0.354 mmol, 97%), was used in the next reaction without further purification. MS (ESI): m/z calcd for C₁₁H₁₃O₆[M+H]⁺ 241.06; found 241.42.

Synthesis of Intermediate Compound 31

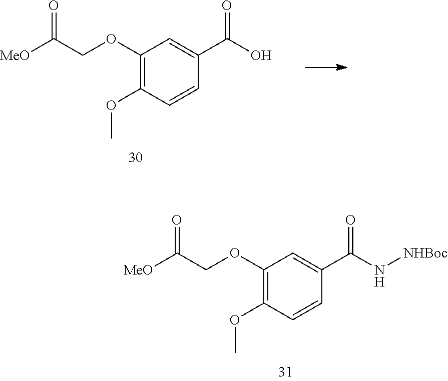 [SMILES: COC(=O)COc1cc(C(=O)O)ccc1OC ; COC(=O)COc1cc(C(=O)NNC(=O)OC(C)(C)C)ccc1OC]

Compound 30 (106 mg, 0.442 mmol) was dissolved in dichloromethane (5 mL) and cooled to 0° C. Oxalyl chloride (118 μL, 1.33 mmol) was added, and the reaction was stirred at 0° C. for 1 h and room temperature for 1 h. The solvent was evaporated and the residue was re-dissolved in dichloromethane (5 mL). NH₂NHBoc (117 mg, 0.884 mmol) and Et₃N (123 μL, 0.884 mmol) were added, and the reaction was stirred at room temperature for 4 h.

Evaporation of the solvent and purification by silica gel chromatography (hexane/ethyl acetate 1:1) gave compound 31 (101 mg, 0.286 mmol, 65%).

¹H NMR (300 MHz, CDCl₃) δ 7.43 (dd, 1H, ArH), 7.33 (d, 1H, ArH), 6.83 (d, 1H, ArH), 4.71 (s, 2H, CH₂C═O), 3.91 (s, 3H, OCH₃), 3.80 (s, 3H, CH₃OC═O), 1.49 (s, 9H, CH₃C); MS (ESI): m/z calcd for C₁₆H₂₃N₂O₇ [M+H]⁺ 355.14; found 355.46.

Synthesis of Intermediate Compound 32

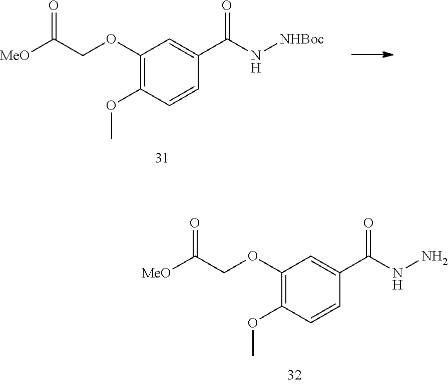 [SMILES: COC(=O)COc1cc(C(=O)NNC(=O)OC(C)(C)C)ccc1OC ; COC(=O)COc1cc(C(=O)NN)ccc1OC]

Compound 31 (170 mg, 0.480 mmol) was dissolved in dichloromethane (5 mL). TFA (200 μL) was added, and the reaction was stirred at room temperature for 2 h. Evaporation of the solvent gave compound 32 (112 mg, 0.439 mmol, 91%). The crude compound was used in the next reaction without further purification. MS (ESI): m/z calcd for C₁₁H₁₅N₂O₅[M+H]⁺ 255.09; found 255.49.

Synthesis of Intermediate Compound 33

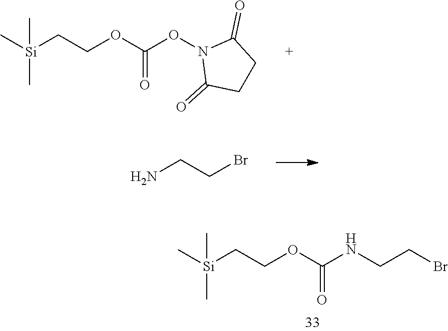

2-Bromo-ethanolamine.HBr (944 mg, 4.6 mmol) and dichloromethane (5 mL) was mixed and Et₃N (0.5 mL, 6.9 mmol) was added, resulting in a slurry mixture. 1-[2-(trimethylsilyl)ethoxycarbonyl oxy]pyrrolidine-2,5-dione (1 g, 3.9 mmol) was dissolved in dichloromethane (5 mL) and added to the mixture, which immediately dissolved the precipitate. The reaction was stirred at room temperature for 5 h and subsequently quenched with water and extracted with dichloromethane (×3). The organic phase was dried over Na₂SO₄, filtrated and solved was removed in vacou. Purification by silica gel chromatography (0-50% ethyl acetate in hexane) gave compound 33 (849 mg, 0.317 mmol, 82%).

¹H-NMR (300 MHz, CDCl₃): δ 5.03 (bs, 1H, NH), 4.13 (t, 2H, CH₂O), 3.53 (t, 2H, NHCH₂), 3.42 (t, 2H, CH₂Br), 0.96 (t, 2H, (CH₃)₃SiCH₂), 0.0 (s, 9H, (CH₃)₃Si)).

Synthesis of Intermediate Compound 34

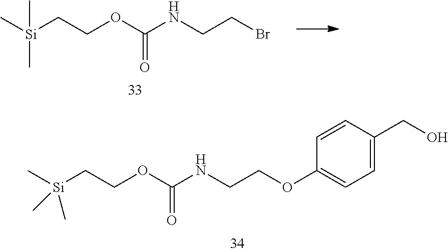 [SMILES: C[Si](C)(C)CCOC(=O)NCCBr ; C[Si](C)(C)CCOC(=O)NCCOc1ccc(CO)cc1]

4-Hydroxybenzylalcohol (248 mg, 2 mmol) was dissolved in anh. dimethylformamide (9.5 mL) and Cs₂CO₃ (caesium carbonate) (651 mg, 2 mmol) was added. The mixture was heated to 75° C. and stirred for 3 h. Compound 33 (536 mg, 2 mmol) was dissolved in anh. dimethylformamide (0.5 mL) and added dropwise to the red/brown suspension. After 4 h, the mixture was cooled to room temperature and stirred overnight. Subsequently, the reaction was quenched with water and extracted with ethyl acetate (×3). The combined organic phase was washed with sat. aqueous NaHCO₃ (sodium hydrogen carbonate) and dried over Na₂SO₄, filtered and concentrated in vacuo. Purification by silica gel chromatography (0-100% ethyl acetate in hexane) gave compound 34 (285 mg, 0.917 mmol, 46%).

¹H-NMR (300 MHz, CDCl₃): δ 7.25 (d, 2H, J=9 Hz, C2H, C6H), 6.83 (d, 2H, J=9 Hz, C3H, C5H), 5.1 (bs, 1H, NH), 4.58 (s, 2H, PhCH₂O), 4.13 (t, 2H, J=8.5 Hz, CH₂OCO), 3.99 (t, 2H, J=5.2 Hz, CH₂O), 3.53 (q, 2H, J=5.2 Hz, NHCH₂), 1.88 (bs, 1H, OH), 0.95 (t, 2H, J=8.5 Hz, SiCH₂), 0.0 (s, 9H, (CH₃)₃Si)

¹³C-NMR (75 MHz, CDCl₃): δ 158.2 (CO), 157.0 (C1), 138.8 (C4), 128.8 (C2, C6), 114.6 (C3, C5), 67.2 (CH₂O), 65.0 (CH₂O), 63.3 (CH₂O), 40.5 (NHCH₂), 17.9 (SiCH₂), −1.4 ((CH₃)₃Si).

Synthesis of Intermediate Compound 35

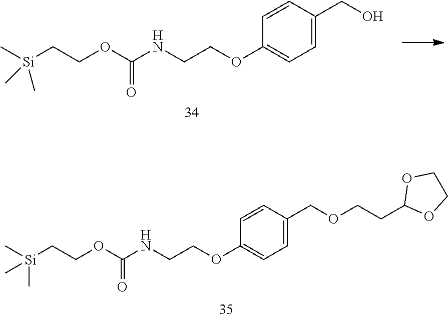 [SMILES: C[Si](C)(C)CCOC(=O)NCCOc1ccc(CO)cc1 ; C[Si](C)(C)CCOC(=O)NCCOc1ccc(COCCC2OCCO2)cc1]

NaH (140 mg, 5.8 mmol) was added to cold anh. tetrahydrofuran (20 mL, under N₂, 0° C.). Compound 34 (908 mg, 2.9 mmol) was dissolved in anh. tetrahydrofuran (0.5 mL) and added dropwise over 15 min. The mixture was stirred at room temperature for 45 min. Then, 3-bromopropionaldehyde ethylene acetal was added dropwise over 10 min. The mixture was stirred at room temperature for 48 h, then filtrated and concentrated in vacuo. Purification by silica gel chromatography (0-60% ethyl acetate in hexane) gave compound 35 (188 mg, 0.457 mmol, 16%).

¹H-NMR (300 MHz, CDCl₃): δ 7.21 (d, 2H, J=8.7 Hz, C3H, C5H), 6.83 (d, 2H, J=8.7 Hz, C2H, C6H), 5.03 (bs, 1H, NH), 4.94 (dq, 1H, CH), 4.41 (s, 2H, PhCH₂O), 4.11 (t, 2H, J=8.4 Hz, CH₂OCO), 3.97 (t, 2H, J=5.1 Hz, CH₂O), 3.95-3.79 (m, 4H, OCH₂CH₂O), 3.56 (t, 2H, J=6.6 Hz, OCH₂CH₂CH), 3.59-3.53 (m, 2H, NHCH₂), 1.94 (dq, 2H, CH₂CH₂CH), 0.95 (t, 2H, J=8.2 Hz, SiCH₂), 0.0 (s, 9H, (CH₃)₃Si)

¹³C-NMR (75 MHz, CDCl₃): δ 191.7, 172.7, 133.5, 130.7, 116.2, 115.5, 104.0, 102.2, 74.2, 68.8, 68.6, 67.2, 66.1, 61.9, 42.9, 35.8, 24.1, 22.5, 19.3, 15.7, 0.0 ((CH₃)₃Si).

Synthesis of Intermediate Compound 36

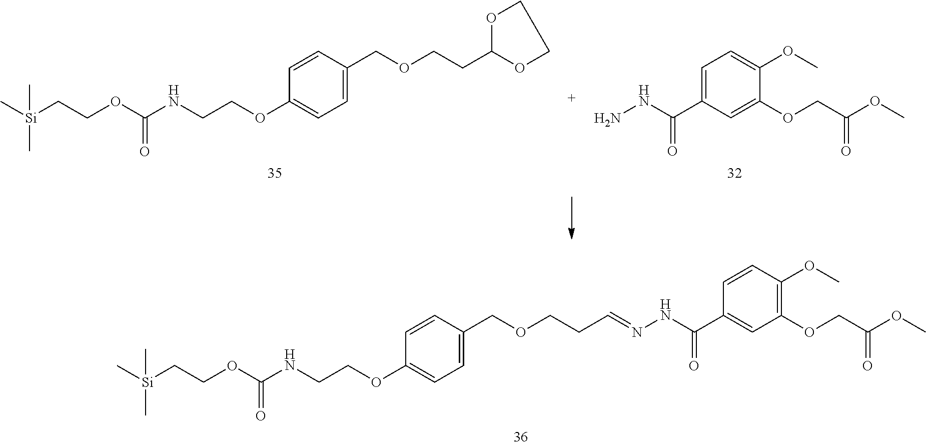

Compound 35 (200 mg, 0.48 mmol) was dissolved in methanol (1.5 mL), and acetic acid (50 μL) was added. The reaction mixture was stirred at 50° C. for 1 h 30 min. Compound 32 was dissolved in methanol (1 mL) and added to the mixture. The solution immediately turned yellow, and after 30 min precipitated was formed. The suspension was filtered and the white powder was washed with methanol (0.5 mL) giving compound 36 (230 mg, 0.381 mmol, 80%).

1H-NMR (300 MHz, DMSO-d₆): δ 11.52 (s, 1H, NNHCO), 8.39 (s, 1H, CHN), 7.65 (d, 2H, J=8.5 Hz, C2H, C6H), 7.60 (dd, 1H, J=2.0 Hz, J=8.5 Hz, C4′H), 7.35 (d, 1H, J=2.0 Hz, C6′H), 7.21 (bt, 1H, NH), 7.12 (d, 1H, J=8.5 Hz, C3′H), 7.0 (d, 2H J=8.5 Hz, C3H, C5H), 4.85 (s, 2H, PhCH₂O), 4.08-4.01 (m, 6H, CH₂CH₂OCO, CH₂OPh, OCH₂CH₂), 3.86 (s, 3H, PhOCH₃, OCH₂COO) 3.8 (m, 2H, CH₂CH₂CHN), 3.69 (s, 3H, COOCH₃), 3.36 (q, 2H, J=5.7 Hz, NHCH₂CH₂O), 0.92 (m, 2H, SiCH₂), 0.0 (s, 9H, (CH₃)₃Si).

¹³C-NMR (75 MHz, DMSO-d₆): δ 168.9 (CO), 163.0 (CO) 159.9, 156.4, 154.5, 151.9, 147.2 (CHN), 146.5, 128.6 (C2,C6), 127.0, 125.5, 121.9 (C4′), 114.8 (C3, C5), 112.7 (C6′), 111.5 (C3′), 66.5 (CH₂OPh), 66.3 (CH₂O), 65.4 (PhCH₂O), 61.6 (CH₂O), 57.8, 55.8 (OCH₃), 54.8 (OCH₂CO), 51.8 (COOCH₃), 39.5 (NHCH2), 17.3 (SiCH₂), −1.8 ((CH₃)₃Si).

Synthesis of Intermediate Compound 37

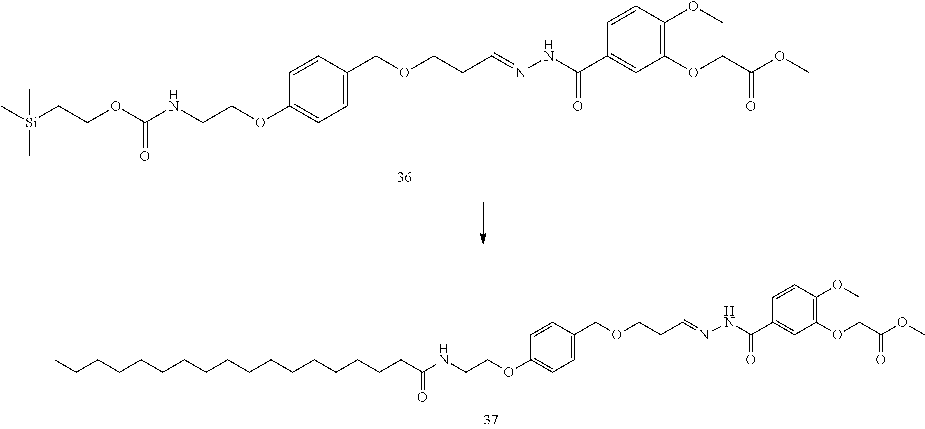

Compound 36 (100 mg, 0.05 mmol) was dissolved in tetrahydrofuran and TBAF (tetrabutylammonium fluoride) (0.2 mL, 1M in tetrahydrofuran, 0.2 mmol) was added. The mixture was heated to 60° C. for 20 h and subsequently cooled to room temperature. Stearic acid (20 mg, 0.07 mmol) was dissolved in tetrahydrofuran (1 mL) and DIPEA (N,N-diisopropylethylamine) (15 μL, 0.086 mmol). TSTU (O—(N-succinimidyl)-1,1,3,3-tetramethyl uranium tetrafluoroborate) (28 mg, 0.09 mmol) was added and the mixture stirred at rt. for 30 min. The activated fatty acid was added to the reaction mixture and stirred at room temperature for 48 h. The excess activated fatty acid was removed by extraction with hexane and the residue was concentrated in vacuo. MS (ESI): m/z calcd for C₄₁H₆₃N₃O₈ [M+H]⁺ 725.46; found 725.84.

Synthesis of Intermediate Compound 38

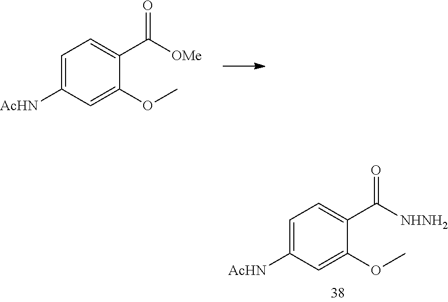 [SMILES: COC(=O)c1ccc(NC(C)=O)cc1OC ; COc1cc(NC(C)=O)ccc1C(=O)NN]

Methyl 4-acetamido 2-methoxy benzoate was synthesised according to Pham et al., J. Med. Chem, 2007, 50(15), 3561-3572.

Methyl 4-acetamido 2-methoxy benzoate (0.201 g, 0.90 mmol) and hydrazine monohydrate (0.440 mL, 9.0 mmol) were dissolved in ethanol and refluxed for 26 h. The reaction mixture was cooled to room temperature, the precipitate was filtered, and dried to give compound 38 (0.157 g, 0.70 mmol, 78%).

¹H NMR (300 MHz, DMSO-d₆): δ 10.14 (s, 1H), 9.08 (s, 1H), 7.71 (d, 1H, J=8.5 Hz), 7.48 (d, 1H, J=1.3 Hz), 7.18 (dd, 1H, J=1.8 Hz, J=8.5 Hz), 3.84 (s, 3H), 2.06 (s, 3H).

Synthesis of Intermediate Compound 39

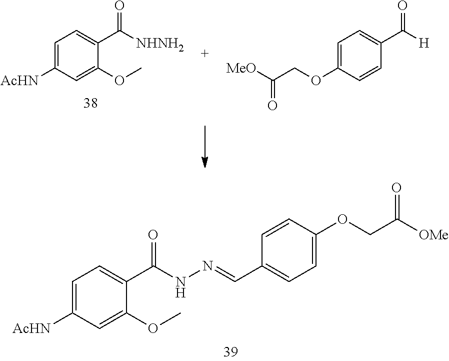 [SMILES: COc1cc(NC(C)=O)ccc1C(=O)NN ; COC(=O)COc1ccc(C=O)cc1 ; COC(=O)COc1ccc(/C=N/NC(=O)c2ccc(NC(C)=O)cc2OC)cc1]

Methyl 2-(4-formylphenoxy)acetate was synthesised according to Karlsson et al., Org. Process. Res. Dev. 2012, 16, 586-594.

Methanol (10 mL) was added to compound 38 (82 mg, 0.367 mmol) and the mixture was gently heated until the compound was fully solubilised. Methyl 2-(4-formylphenoxy)acetate (65 mg, 0.334 mmol) was added and the reaction mixture was refluxed for 1 h. After cooling of the reaction mixture, the formed precipitate was filtered and dried to give compound 39 as a white solid (130 mg, 0.325 mmol, 89%).

¹H NMR (300 MHz, DMSO-d₆): δ 11.17 (s, 1H), 10.21 (s, 1H), 8.31 (s, 1H), 7.66 (t, 3H), 7.53 (d, 1H, J=1.4 Hz), 7.22 (dd, 1H, J=8.5 Hz, J=1.6 Hz), 7.01 (d, 2H), 4.86 (s, 2H), 3.88 (s, 3H), 3.71 (s, 3H), 2.07 (2, 3H). MS (ESI): m/z calcd for C₂₀H₂₁N₃O₆: 400.14 [M+H]⁺; found 400.04.

Synthesis of Intermediate Compound 40

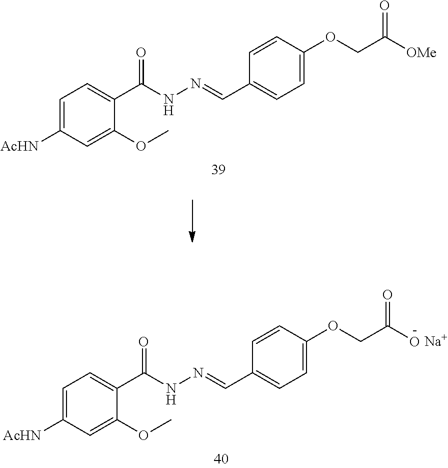

Compound 39 (50 mg, 0.124 mmol) was dissolved in tetrahydrofuran/methanol/water 2/1/1 (8 mL). NaOH (5M, 100 μL) was added and the reaction was stirred at room temperature for 30 min. The solvent was evaporated and tetrahydrofuran (10 mL) was added. The formed precipitate was isolated by centrifugation and dried to give compound 40 (45 mg, 0.110 mmol, 88%).

¹H NMR (300 MHz, DMSO-d₆): δ 8.13 (s, 1H), 7.52 (d, 2H, 1H, J=8.9 Hz), 7.22 (d, 1H, J=8.8 Hz), 7.13 (s, 1H), 6.82 (d, 1H, J=8.5 Hz), 6.77 (d, 2H, J=8.8 Hz), 4.12 (s, 2H), 3.61 (s, 3H), 1.85 (s, 3H). MS (ESI): m/z calcd for C₁₉H₁₉N₃O₆: 386.15 [M+H]⁺; found 386.12.

Synthesis of Intermediate Compound 41

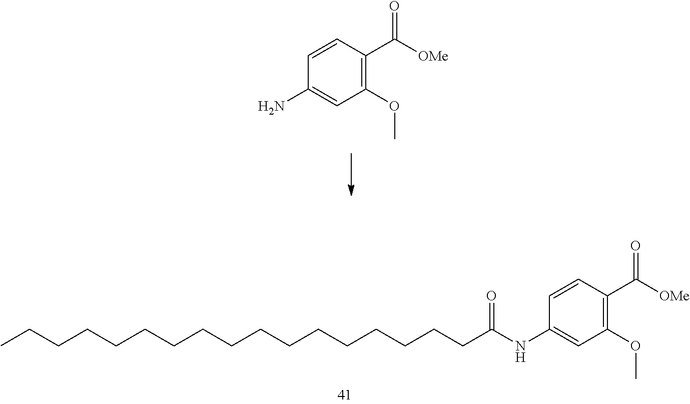 [SMILES: COC(=O)c1ccc(N)cc1OC ; CCCCCCCCCCCCCCCCCC(=O)Nc1ccc(C(=O)OC)c(OC)c1]

Oxalyl chloride (0.9 mL, 10.5 mmol) was added to stearic acid (1.0 g, 3.52 mmol) in dichloromethane (10 mL). After stirring the suspension at room temperature for 1 h the starting materials were dissolved and the reaction was finished. The solvent was evaporated and the activated acid was re-dissolved in dichloromethane (10 mL) where after methyl 4-amino 2-methoxy benzoate (0.76 g, 4.22 mmol) was added. The reaction was stirred at room temperature overnight. After evaporation, the crude was purified by silica gel chromatography (dichloromethane/methanol 25:1) to give compound 41 (1.33 g, 2.97 mmol, 84%).

¹H NMR (300 MHz, CDCl₃): δ 7.80 (d, J=8.5 Hz, 1H), 7.70 (s, 1H), 6.78 (dd, J=8.5 Hz, J=2.0 Hz, 1H), 3.91 (s, 3H), 3.86 (s, 3H), 2.37 (m, 2H), 1.72 (m, 2H), 1.25 (m, 28H), 0.88 (m, 3H).

Synthesis of Intermediate Compound 42

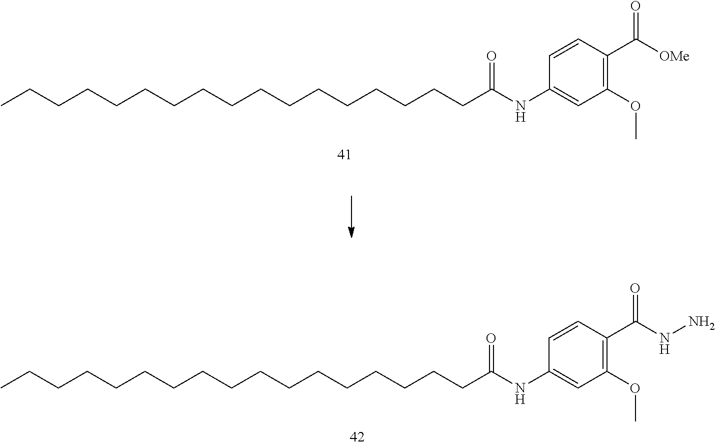 [SMILES: CCCCCCCCCCCCCCCCCC(=O)Nc1ccc(C(=O)OC)c(OC)c1 ; CCCCCCCCCCCCCCCCCC(=O)Nc1ccc(C(=O)NN)c(OC)c1]

Hydrazine monohydrate (0.16 mL, 3.35 mmol) was added to compound 41 (0.15 g, 0.335 mmol) in ethanol (10 mL). The reaction mixture was refluxed overnight and subsequently cooled to room temperature. The product precipitated and was isolated by filtration and dried, to give compound 42 (0.122 g, 0.273 mmol, 81%).

¹H NMR (300 MHz, CDCl₃): δ 8.89 (s, 1H), 8.12 (d, J=8.5 Hz, 1H), 7.91 (s, 1H), 7.42 (s, 1H), 6.74 (dd, J=8.5 Hz, J=1.8 Hz, 1H), 2.38 (t, J=7.4 Hz, 2H), 1.77-1.67 (m, 2H), 1.24 (s, 28H), 0.87 (t, J=6.4 Hz, 3H).

Synthesis of Intermediate Compound 43

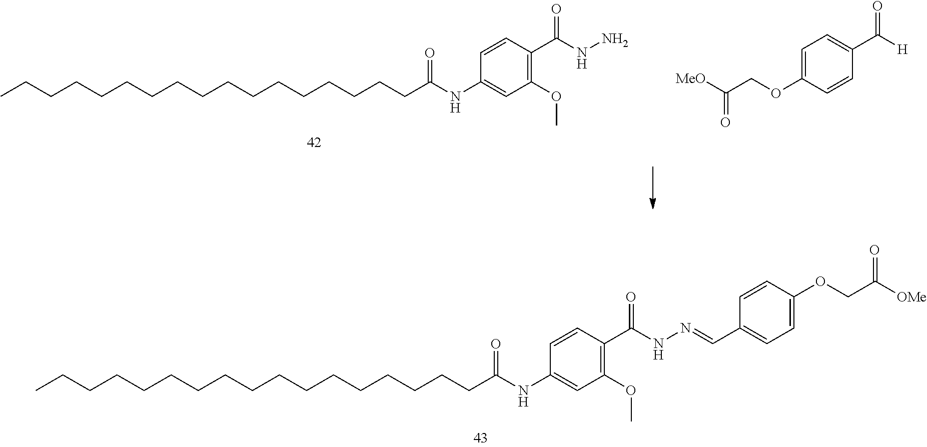 [SMILES: CCCCCCCCCCCCCCCCCC(=O)Nc1ccc(C(=O)NN)c(OC)c1 ; COC(=O)COc1ccc(C=O)cc1 ; CCCCCCCCCCCCCCCCCC(=O)Nc1ccc(C(=O)N/N=C/c2ccc(OCC(=O)OC)cc2)c(OC)c1]

Compound 42 (35 mg, 0.078 mmol) was dissolved in ethyl acetate (10 mL) by heating the mixture to 60° C. Methyl 2-(4-formylphenoxy)acetate (14 mg, 0.071 mmol) followed by acetic acid (3 drops) were added and the reaction was refluxed for 30 min. The solvent was evaporated and the residue was purified by silica gel chromatography (dichloromethane/methanol, gradient from 50:1 to 25:1) to give compound 43 (31 mg, 0.050 mmol, 64%).

¹H NMR (300 MHz, CDCl₃): δ 11.15 (s, 1H), 10.13 (s, 1H), 8.32 (s, 1H), 7.70-7.58 (m, 4H), 7.22 (dd, J=8.5 Hz, J=1.6 Hz, 1H), 7.02 (d, J=8.8 Hz, 2H), 4.86 (s, 2H), 3.88 (s, 3H), 3.71 (s, 3H), 2.33 (t, J=7.2 Hz, 2H), 1.60-1.56 (m, 2H), 1.23 (s, 28H), 0.85 (t, J=6.5 Hz, 3H).

Synthesis of Intermediate Compound 44

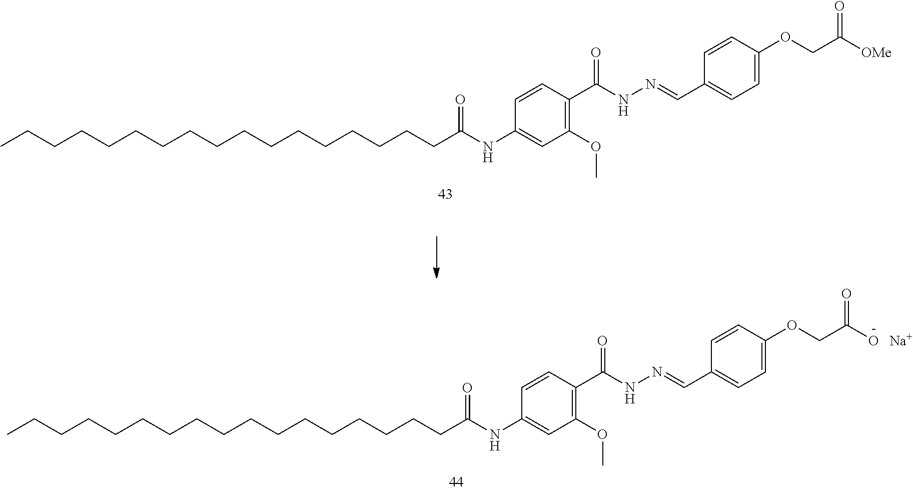

Compound 43 (10.5 mg, 0.017 mmol) was dissolved in tetrahydrofuran (1 mL). Methanol (1 mL) followed by water (1 mL) were added and the solution turned milky. NaOH (5M, 100 μL) was added and the reaction was stirred for 30 min. The formed precipitate was filtered and dried to give compound 44 (9.0 mg, 0.0148 mmol, 89%).

¹H NMR (300 MHz, CDCl₃): δ 11.07, (s, 1H), 10.12 (s, 1H), 8.29 (m, 2H), 7.69 (d, J=8.4 Hz, 1H), 7.56 (m, 3H), 7.21 (dd, J=8.4 Hz, J=1.7 Hz, 1H), 6.83 (d, J=8.9 Hz, 1H), 4.09 (s, 2H), 3.89 (s, 3H), 2.32 (t, J=7.3 Hz, 2H), 1.66-1.55 (m, 2H), 1.24 (s, 28H), 0.85 (t, J=6.4 Hz, 3H).

### Example 4: Synthesis of Lys^{b29}N^{ε}-Octadecanoyl Human Insulin (Reference Insulin Conjugated to Inactivator Via Non-Hydrolysable Binding)

DIPEA (41 μL, 0.23 mmol) and TSTU (30.5 mg, 0.10 mmol) were added to a stirred solution of octadecanoic acid (22 mg, 0.078 mmol) in THF (6 mL). The reaction was monitored by TLC and full conversion to the active-ester was observed after 3 h. Human insulin (100 mg, 0.0178 mmol) was dissolved in 3 mL 0.1 M Na₂CO₃ and the pH was adjusted to 10.5 with 0.1M NaOH. The active ester was added drop-wise under gently stirring and the pH was adjusted to 10.5 during the addition. The reaction was followed by LC-MS and a 58% conversion to product was observed after 15 min. The pH was lowered to 4-5, water was added and the mixture was lyophilised. The crude white powder was purified by reversed-phase HPLC (C4 column, water/acetonitrile/0.1% TFA), and quantified by UPLC-MS (C18 column, acetonitrile/water/formic acid).

MS (ESI) calcd. for C₂₇₅H₄₁₇N₆₅O₇₈S₆: 6074.10 [M+H]⁺ , 2025.71 [M+3H]³⁺, 1519.53 [M+4H]⁴⁺, 1215.83 [M+5H]⁵⁺, 1013.36 [M+6H]⁶⁺; found 2025.79, 1519.54, 1216.00, 1013.51.

### Example 5: Synthesis of Insulin Conjugate According to the Invention (Conjugate 2)

General Procedure:

Dimethylformamide (0.5 mL) and 15-crown-5 (10 equiv) were added to compound 40 or 44 (1 mg) and stirred for 1 h until the starting materials were dissolved. TSTU (1.3 equiv) in dimethylformamide (0.1 mL) was added and the reaction was stirred at room temperature for 10 min, followed by dropwise addition to a gently stirred solution of human insulin (2 equiv) in DMSO containing Et₃N (100 equiv). After 10 min, the reaction mixture was analysed by LC-MS confirming the formation of the insulin conjugate. Acetonitrile (0.5 mL) and water (0.5 mL) were added and pH was adjusted to 7.5 by addition of acetic acid. Purification by RP-Flash Chromatography (Isolera One™, Biotage) on a 10 g C4 column using a gradient of water, 0.1% formic acid towards acetonitrile 0.1% formic acid. The pH of each fraction was adjusted to around 7.5 using aqueous NH₃. The pure fractions were pooled, acetonitrile was evaporated and pH was adjusted to 8 by aqueous NH₃, followed by lyophilisation to give the insulin conjugate as white powder.

Insulin Conjugate 1

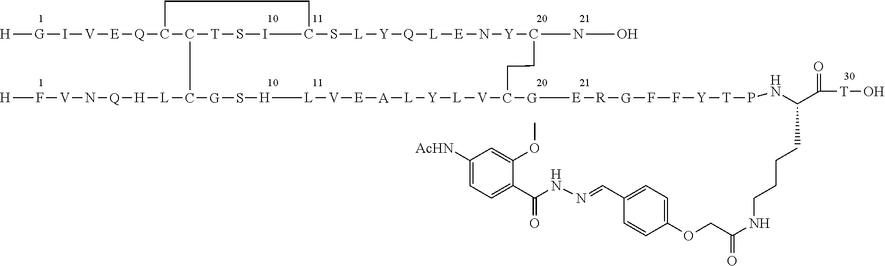

MS (ESI): m/z calcd for C₂₇₆H₄₀₀N₆₈O₂S₆: 6175.99 [M+H]⁺, 1544.76 [M+4H]⁴⁺, 1236.01 [M+5H]⁵⁺, 1030.17 [M+6H]⁶⁺; found 1544.92, 1235.86, 1030.11.

Insulin Conjugate 2

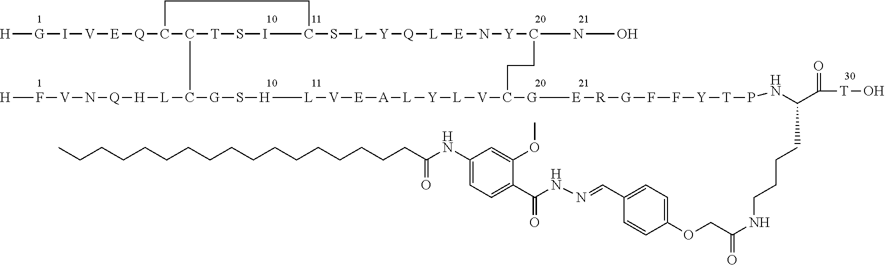

MS (ESI): m/z calcd for C₂₉₂H₄₃₂N₈₀O₂S₆: 6399.42 [M+H]⁺, 1600.86 [M+4H]⁴⁺, 1280.89 [M+5H]⁵⁺, 1067.58 [M+6H]⁶⁺; found 1601.03, 1281.10, 1067.67.

### Example 6: In Vitro Glucose Sensing Evaluation (LC-MS)

The aim with this example is to evaluate the reactivity of each linker towards glucose.

General Protocol:

1-1.5 mg of linker was dissolved in 100 μL DMSO. Immediately after solvation, 5 μL was added to 995 μL phosphate buffer pH 7 containing 1000 equiv glucose. The mixture was heated to 37° C. and analysed continuously by LC-MS

| Linker | Linker-glucose observed (h) |
|---|---|
| 1 | 8.5 |
| 2 | ND after 96 h |
| 4 | ND after 96 h |
| 5 | ND after 96 h |
| 6 | ND after 96 h |
| 7 | ND after 96 h |
| 8 | 72 |
| 9 | 24 |
| 10 | 20 |
| 11 | 20 |
| 12 | 15 |
| 13 | 15 |
| 14 | 17 |
| 15 | 5 |
| 16 | 48 |
| 17 | 48 |
| 18 | ND after 96 h |
| 19 | 48 |

Results and Discussion:

From example 2, the following structure-glucose reaction relationship of the linker molecules seems reasonable:

Starting from the general formula

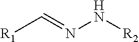

the R₁ component is flexible, but an aromatic ring spaced with an alkane chain to the hydrazone seems to be important for the rate of linker-glucose binding. The alkyl chain can also be an alkane ether. The R₂ component should preferably be an aromatic ring with donating groups in the ortho and/or para position.

The linker with the highest rate of glucose binding in example 6 is linker 15, which forms a linker-glucose within 5 hours.

### Example 7: In Vitro Glucose Sensing at Different Glucose Concentration

The aim with the example is to evaluate the reaction rate of three different linkers at various glucose concentrations i.e. their ability to hydrolyse and react with glucose to form a linker glucose conjugate.

Procedure 1:

1.4 mg of linker 1 ((E)-N′-(3-(benzyloxy)propylidene)-4-methoxybenzohydrazide) was dissolved in 100 μL DMSO. 10 μL of the DMSO stock solution was added to 990 μL 1×PBS buffer pH 7.4, containing 1000 or 5000 equiv glucose, to give a final concentration of 0.42 mM of linker 1. The solutions were heated to 37° C. and analysed at different time points from 0 to 48 h by UPLC-MS (C18 column, acetonitrile/water/formic acid). The linker-glucose compound was analysed as percentage of the full conversion of the reaction.

TABLE 1
Linker 1 (% linker-glucose)

| | 1000 equiv | 5000 equiv |
|---|---|---|
| Time (h) | glucose | glucose |
| 0 | 0 | 0 |
| 0.75 | n.d. | 3 |
| 1 | n.d. | 6 |
| 1.25 | n.d. | 7 |
| 1.5 | 3 | 10 |
| 1.75 | n.d. | 10 |
| 2 | 3 | 14 |
| 2.5 | n.d. | 18 |
| 3 | 5 | n.d. |
| 3.17 | 4 | n.d. |
| 3.5 | 4 | n.d. |
| 3.75 | 4 | n.d. |
| 4 | 5 | n.d. |
| 4.5 | 6 | n.d. |
| 4.75 | 6 | n.d. |
| 5 | 6 | n.d. |
| 5.5 | 6 | n.d. |
| 6 | 9 | n.d. |
| 7 | 9 | 49 |
| 24 | 29 | n.d. |
| 48 | 70 | n.d. |

Procedure 2:

1.3 mg and 1.4 mg of linker 14 ((E)-N′-(2-(benzyloxy)ethylidene)-4-methoxybenzohydrazide) and linker 15 ((E)-N′-(2-(benzyloxy)ethylidene)-2-hydroxy-4-methoxybenzohydrazide), respectively, was dissolved in 100 μL DMSO. 10 μL of each stock solution was added to 990 μL 1×PBS buffer pH 7.4 containing 1000, 5000 or 10,000 equiv glucose, to give a final linker concentration of 0.42 mM. The solutions were heated to 37° C. and analysed at different time points from 0 to 72 h by UPLC-MS (C18 column, acetonitrile/water/formic acid). The linker-glucose compounds were analysed as percentage of the full conversion of the reaction.

TABLE 2
Linker 14 (% linker-glucose)

| | 1000 equiv | 5000 equiv | 10,000 equiv |
|---|---|---|---|
| Time (h) | glucose | glucose | glucose |
| 0 | 0 | 0 | 0 |
| 6 | n.d. | n.d. | 3 |
| 24 | 7 | 16 | 39 |
| 54 | 11 | 30 | 62 |
| 72 | 17 | 50 | 87 |

TABLE 3
Linker 15 (% linker-glucose)

| | 1000 equiv | 5000 equiv | 10,000 equiv |
|---|---|---|---|
| Time (h) | glucose | glucose | glucose |
| 0 | 0 | 0 | 0 |
| 1 | n.d. | 2 | 9 |
| 2 | 1 | 6 | 15 |
| 4 | 3 | 12 | 33 |
| 6 | 5 | 21 | 44 |
| 24 | 21 | 68 | 89 |
| 48 | 35 | 98 | 99 |

Result and Discussion:

In all three examples the reaction rate, i.e. the amount of formed linker glucose conjugate, correlates with increasing glucose concentrations.

### Example 8: In Vitro Glucose Sensing Evaluation of Insulin Conjugate

The aim with this example is to evaluate the hydrolysability of the linker attached to insulin, in the presence of glucose.

General Protocol:

1.0 mg of the insulin conjugate was dissolved in 100 μL DMSO. 10 μL was added to 490 μL 1×PBS buffer pH 7.4, containing 50 000 equiv glucose, to give a final concentration of 32 μM. The sample was incubated at 37° C. and analysed at 1, 24, 48 and 72 h by HPLC (C18 column, acetonitrile/water/formic acid).

| | Insulin conjugate 2 (%) | | | | |
|---|---|---|---|---|---|
| | Start | 1 h | 24 h | 48 h | 72 h |
| No glucose | 100 | 92 | 89 | 88 | 87 |
| 50 000 equiv | 100 | 92 | 76 | 68 | 58 |
| glucose | | | | | |

Result and Discussion:

In the absence of glucose, the insulin conjugate is hydrolysed, the equilibrium is stabilised and remains the same throughout the experiment. When glucose is present, the dynamic equilibrium is shifted from the insulin conjugate towards insulin with a hydrolysed linker, which indicates glucose sensitivity of the linker.

### Example 9: In Vitro Insulin Receptor B (INSRb) Functional Assay

The purpose of this example is to test the in vitro potency on the insulin B receptor.

General Protocol:

The PathHunter INSRb functional assay kit (DiscoverX) with a 1×PBS buffer containing 0.1% BSA (Bovine Serum Albumin) pH 7.4, instead of the manufacturing buffer, was used.

| Compound | EC50 (nM) |
|---|---|
| Human insulin | 0.10 |
| Insulin conjugate 1 | 0.11 |
| Insulin-C18 | 31 |
| Insulin conjugate 2 | 8.2 |

Result and Discussion:

The potency of the insulin conjugate 1 (insulin without inhibitor) is similar to the potency of human insulin. The potency of the insulin conjugate 2 is 100-fold lower than that of human insulin and the potency of insulin-C18 is 300 fold lower than the potency of human insulin.

### Example 10: In Vivo scITT of Insulin-C18 in Lean Rats

The aim with this example is to evaluate human insulin conjugated with a C18 fatty acid and its ability to interact with albumin and reduce insulin activity, measured by scITT in lean rats. Blood glucose concentrations were measured before and at five time-point after subcutaneous administration of vehicle, 5 U of insulin-C18 or 0.5 U of human insulin (n=4).

| | Blood glucose % of vehicle (±SEM) | | | | | |
|---|---|---|---|---|---|---|
| | 0 min | 60 min | 120 min | 180 min | 240 min | 300 min |
| Human | 95 ± 3 | 64 ± 4 | 55 ± 6 | 66 ± 7 | 76 ± 4 | 76 ± 4 |
| insulin | | | | | | |
| Insulin- | 107 ± 3 | 102 ± 2 | 101 ± 2 | 96 ± 3 | 99 ± 3 | 99 ± 5 |
| C18 | | | | | | |

Result and Discussion:

The result indicates that insulin-C18 have a strong interaction with albumin and thereby eliminate the action of insulin during the time of the measurement.

1. A conjugate of the formula P-L-I, wherein P is a peptide hormone effecting the metabolism of carbohydrates in vivo, L is a hydrolysable linker molecule consisting of Lₚ and Lᵢ, and I is a molecule capable of inactivating or inhibiting the effect of the peptide hormone P on the metabolism of carbohydrates in vivo, wherein
a. the linker molecule L is hydrolysable in vivo, such that the conjugate P-L-I and the conjugate parts P-Lₚ and Lᵢ-I exist in a dynamic equilibrium in vivo where the conjugate P-L-I exists in molar excess of at least one of the conjugate parts P-Lₚ and Lᵢ-I, and further characterised in that
b. at least one of the conjugate parts P-Lₚ and Lᵢ-I binds covalently to glucose, whereby the concentration of P that is not bound to I increases in vivo when the concentration of glucose increases in vivo, or, alternatively further wherein the hydrolysis of the hydrolysable linker L is being promoted by glucose.

2. Conjugate according to claim 1, wherein the reactant P-Lₚ binds covalently to glucose.

3. Conjugate according to claim 1, wherein P is insulin or an insulin analogue.

4. Conjugate according to claim 1, wherein I is an agent capable of inhibiting the active site of P.

5. Conjugate according to claim 1, wherein I is an agent capable of clustering multiple conjugates of the formula P-L-I in vivo.

6. Conjugate according to claim 1, wherein I is an agent capable of binding to serum albumin.

7. Conjugate according to claim 1, wherein I comprises the structure A, where A is selected among 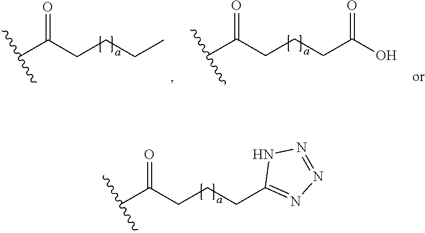
and a is at least 10.

8. Conjugate according to claim 1, wherein L is selected among hydrazones, O,O-acetals, N,O-acetals, N,N-acetals, S,N-acetals including thiazolidines and thiazolines, or S,S-acetals including dithiolanes, and their derivatives.

9. Conjugate according to claim 1, wherein L is of the general formulae 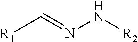
wherein
R₁ comprises I or P, preferably attached to an aromatic moiety, and
R₂ comprises P or I.

10. Conjugate according to claim 7, wherein L is of the general formulae 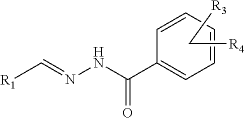
wherein
R₁ comprises an aromatic moiety to which I or P is attached, and
R₃ is one or more electron-donating groups, and
R₄ comprises P or I.

11. Conjugate according to claim 1 for the treatment or prophylactic treatment of a human or animal subject.

12. Conjugate according to claim 1 for the treatment diabetes in a human or animal subject.

13. Conjugate according to claim 1 for the treatment of diabetes mellitus in a human or animal subject, the treatment comprising administering the conjugate in a frequency of 2 or less administrations per day.

14. Conjugate according to claim 1 for the treatment of diabetes mellitus in a human or animal subject, the treatment comprising administering the conjugate in a frequency of 1 or less administrations per day.

15. Pharmaceutical or veterinary composition comprising a conjugate according to claim 1 and at least one pharmaceutical or veterinary excipient.
